(19) United States
(12) Patent Application Publication
(10) Pub. No.: US 2026/0240612 A1
(43) Pub. Date: Aug. 20, 2026

(54) SYSTEMS AND METHODS FOR A LOW-CONDUCTIVITY AND HIGH PERMEABILITY BASED TARGET INDUCTIVE ENCODING

(71) Applicant: VICARIOUS SURGICAL INC., Waltham, MA (US)

(72) Inventors: Daniel Fay, Somerville, MA (US); Orod Bavar, Westborough, MA (US); Ryan Fish, Playa Vista, CA (US); Tyler McCollum, Somerville, MA (US)

(21) Appl. No.: 19/469,843

(22) PCT Filed: Apr. 1, 2024
(86) PCT No.: PCT/US2024/022562

§ 371 Date: Sep. 26, 2025

Related U.S. Application Data
(60) Provisional application No. 63/456,390, filed on Mar. 31, 2023.

(51) Int. Cl. A61B 34/30; A61B 34/20

(52) U.S. Cl. CPC A61B 34/30; A61B 34/20; A61B 2034/2055; A61B 2034/2059; A61B 2034/302

(57) ABSTRACT

Systems and methods are disclosed herein for determining an angular position of a rotary joint in a robotic system. The systems and methods use a coil coupled to a first portion of the rotary joint at a first location. A target having high permeability and low conductivity properties is coupled to a second portion of the rotary joint. An oscillator circuit is communicatively coupled to the coil and a computing unit is communicatively coupled to the oscillator circuit. The computing unit includes a processor configured to or programmed to determine a position of the rotary joint based at least in part on a frequency associated with a signal received from the oscillatory circuit. The frequency being based at least in part on a positional relationship between the target and the coil.

### CROSS REFERENCE TO RELATED APPLICATIONS

This present application claims the benefit of U.S. Provisional Application Ser. No. 63/456,390, filed Mar. 31, 2023, the contents all of which are incorporated herein by reference.

### BACKGROUND OF THE DISCLOSURE

Surgical robotic systems permit a user (also described herein as an “operator” or a “user”) to perform an operation using robotically-controlled instruments to perform tasks and functions during a procedure. Position sensors are used within one or more robotic arms of the surgical robotic systems to output signals to a processor that can be used to resolve the position of the one or more robotic arms within a cavity of a patient during a procedure.

Position sensors allow the surgical robotic system to determine the position of the joints of the one or more robotic arms, in turn the position of the joints of the one or more robotic arms is made available to a user. One conventional type of position sensor is known as a Hall effect sensor.

### SUMMARY

A surgical robotic system is presented. The surgical robotic system can determine an angular position of a joint of a robotic arm. The surgical robotic system can include a first coil mounted to the robotic joint at a first location, a second coil mounted to the robotic joint at a second location, a target attached to the joint of the robotic arm, an oscillator circuit communicatively coupled to the first coil and the second coil, and a computing unit communicatively coupled to the oscillator circuit. The computing unit includes a processor. The processor can be configured to or programmed to read the one or more instructions stored in memory to determine an angular position of the joint of the robotic arm based at least in part on a first frequency associated with a first signal received from the oscillatory circuit. The first frequency being based at least in part on a positional relationship between the target and the first coil and the second coil. The embodiments taught herein of one or more coils in combination with a low-conductivity and high permeability target and one or more oscillatory circuits are able to sense axial distance, as well as rotational or translational position of the target relative to the sense coils.

In some embodiments the target can comprise a first material that has an absolute permeability that is greater than 100 Henry per meter (H/m) and a second material that has a conductivity that is less than 2×10\hat{ }−2 Siemens per meter (S/m).

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 schematically depicts an example surgical robotic system in accordance with some embodiments.

FIG. 2A is an example perspective view of a patient cart including a robotic support system coupled to a robotic subsystem of the surgical robotic system in accordance with some embodiments.

FIG. 2B is an example perspective view of an example operator console of a surgical robotic system of the present disclosure in accordance with some embodiments.

FIG. 3A schematically depicts an example side view of a surgical robotic system performing a surgery within an internal cavity of a subject in accordance with some embodiments.

FIG. 3B schematically depicts an example top view of the surgical robotic system performing the surgery within the internal cavity of the subject of FIG. 3A in accordance with some embodiments.

FIG. 4A is an example perspective view of a single robotic arm subsystem in accordance with some embodiments.

FIG. 4B is an example perspective side view of a single robotic arm of the single robotic arm subsystem of FIG. 4A in accordance with some embodiments.

FIG. 5 is an example perspective front view of a camera assembly and a robotic arm assembly in accordance with some embodiments.

FIG. 6A is an example perspective view of a left hand controller for use in an operator console of a surgical robotic system in accordance with some embodiments.

FIG. 6B is an example perspective view of a right hand controller for use in an operator console of a surgical robotic system in accordance with some embodiments.

FIG. 7A is an example perspective view of a left hand controller for use in an operator console of a surgical robotic system in accordance with some embodiments.

FIG. 7B is an example perspective view of a right hand controller for use in an operator console of a surgical robotic system in accordance with some embodiments.

FIG. 8A is an example perspective view of a left hand controller for use in an operator console of a surgical robotic system in accordance with some embodiments.

FIG. 8B is an example perspective view of a right hand controller for use in an operator console of a surgical robotic system in accordance with some embodiments.

FIG. 9A is an example differential arrangement of position sensors with a target and coil sensors in a robotic joint, in accordance with some embodiments.

FIG. 9B is an example differential arrangement of position sensors with a target and coil sensor in a robotic joint, in accordance with some embodiments.

FIG. 10A is a side view of an example position sensor with a target, redundant coil sensors, and a background in a robotic joint, in accordance with some embodiments.

FIG. 10B is a top down view of an example position sensor with a target, redundant coil sensors, and background in a robotic joint, in accordance with some embodiments.

FIG. 11 is a quadrature arrangement of a target as well as sensor coils used in a position sensor for robotic joints with near or greater than 360 degrees of rotation, in accordance with some embodiments.

FIG. 12A is an example schematic of a sense coil and an oscillator circuit that is electrically coupled to the sense coil and where the oscillator circuit outputs an encoder signal associated with a position of a robotic joint, in accordance with some embodiments.

FIG. 12B is an example schematic of a sense coil and an oscillator circuit that is electrically coupled to the sense coil and where the oscillator circuit outputs an encoder signal associated with a position of a robotic joint, in accordance with some embodiments.

FIG. 12C is an example switching circuit connecting two exemplary oscillator circuits, in accordance with some embodiments.

FIG. 13 is an example encoder signal output in relation to a position sensor associated with a position of a robotic joint, in accordance with some embodiments.

FIG. 14 depicts a relationship between a prior art high conductivity and low permeability target with a plastic or air background in a position sensor of a robotic joint, in accordance with some embodiments.

FIG. 15A depicts a relationship between a low conductivity and high permeability target with a plastic or air background in a position sensor of a robotic joint, in accordance with some embodiments.

FIG. 15B depicts a relationship between a low conductivity and high permeability target with a background having a high conductivity and low permeability in a position sensor of a robotic joint, in accordance with some embodiments.

FIG. 16 depicts a target made of two materials one of which can have a low conductivity and high permeability while the other material has a high conductivity and low permeability, in accordance with some embodiments.

FIG. 17 is a flow diagram for generating an encoder value associated with a position of a robotic joint, in accordance with some embodiments.

FIG. 18 is an example flowchart corresponding to determining a position of a robotic joint using an inductive sensing method, in accordance with some embodiment.

FIG. 19 schematically depicts an example computing module of the surgical robotic system in accordance with some embodiments.

### DETAILED DESCRIPTION

As noted above, position sensors enable an operator of a surgical robotic system to determine precisely where a robotic arm, and therefore surgical instruments are within the cavity of a patient. However, these position sensors are oftentimes designed using coupled coils, for example, two coils sharing a magnetic circuit. Coupled coils are limited by their size constraint in that there needs to be sufficient space for both a primary coil and a secondary coil of the coupled coils, as well as any passive components used to create a resonant circuit, which is used to encode the position of the robotic arms as they move. This in turn has the effect of limiting the size of the robotic arms, and limiting the resolution of the positioning information that is encoded by the robotic arms.

As an alternative, a sense coil and target design can be used with smaller robotic arms and that provide a greater resolution of the position of the robotic arms. The sense coil and conductive target work together to decrease the inductance of the sense coil, which can then be measured to determine the position of one portion of a joint of the one or more robotic arms to another portion of the one or more robotic arms. This requires the inductance of the sense coil to be rather large such that a measurement can be made and needs to stay large enough even as the inductance is reduced in order to continue to make measurements as the inductance diminishes. The size of the sense coil is directly related to the resolution of positioning information associated with the position of the one or more robotic arms as they are being moved. Needless to say, both of these approaches result in significant difficulties when in a highly space-constrained environment, for example, one or more robotic arms operating in a cavity of a patient.

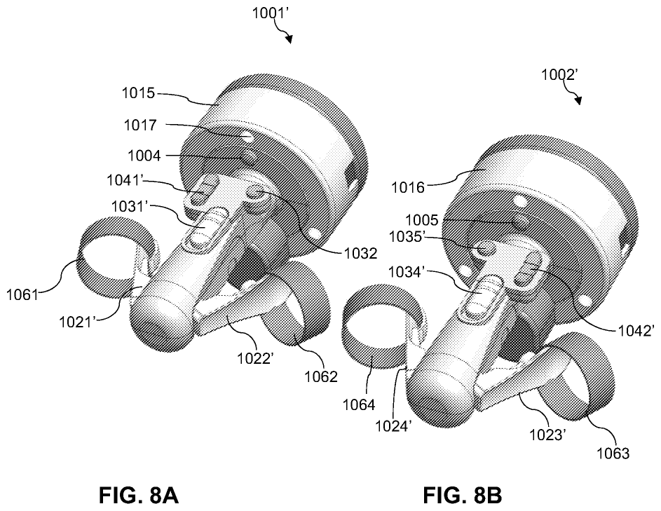

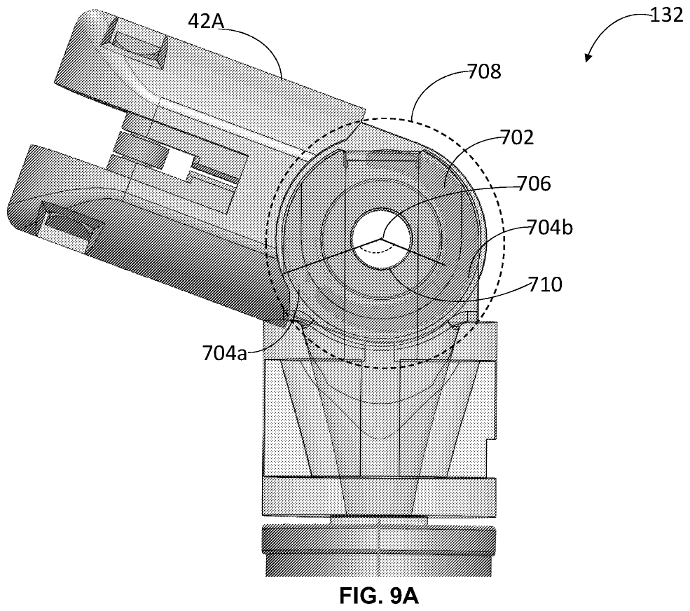

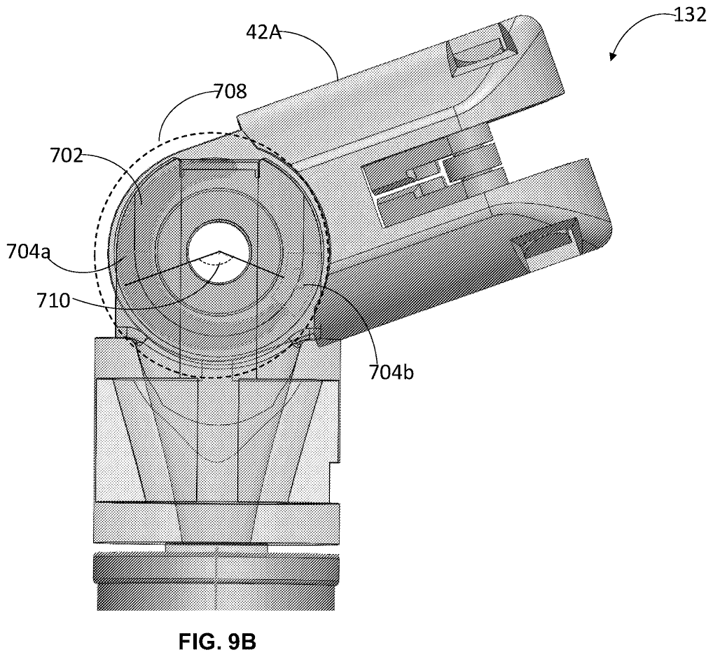

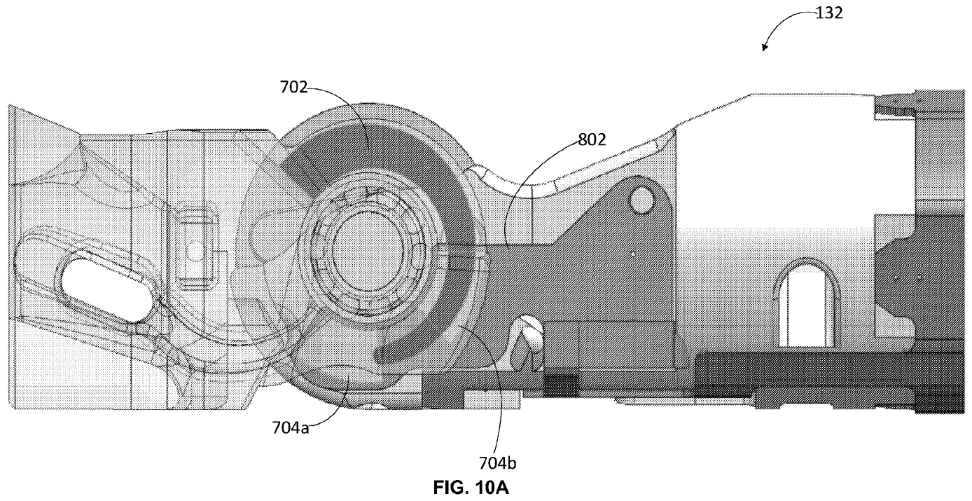

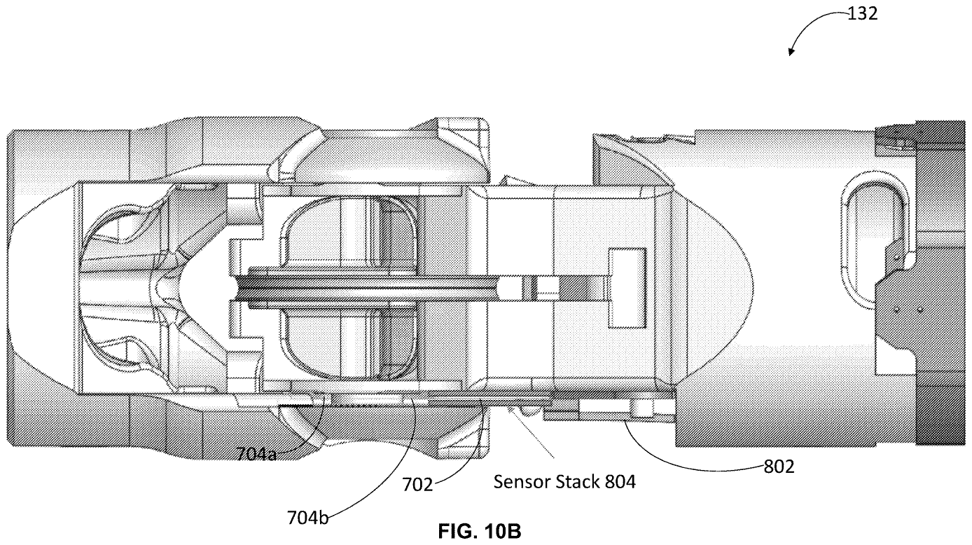

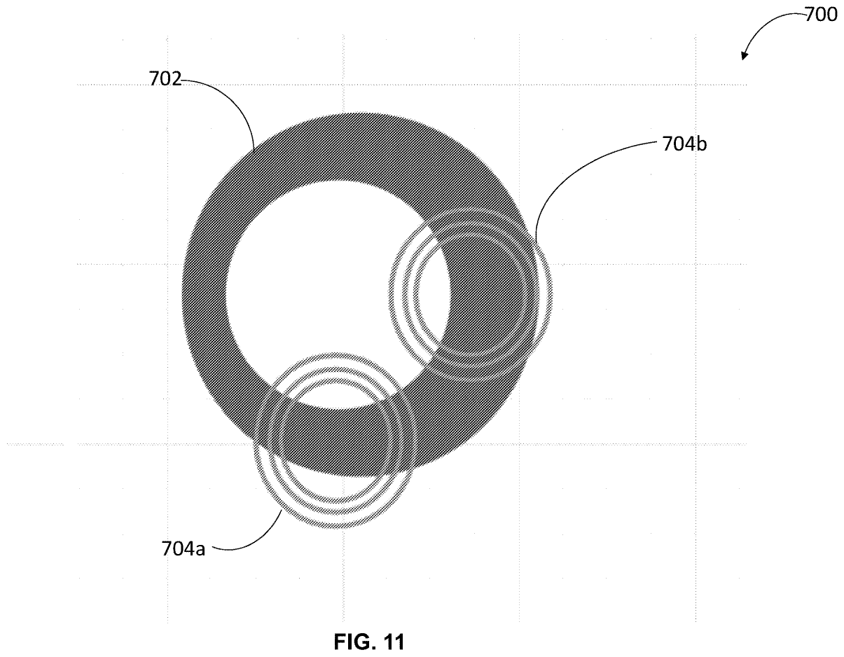

In order to facilitate explanation of the inductive encoding technology disclosed herein, an application in robotics is disclosed, however the inductive encoding technology disclosed herein can also be applied in the areas of user interfaces or motor encoders subject to wet conditions, or dirt and debris infiltration such as industrial environments. The disclosed inductive encoding technology can also be applied in highly space constrained (particularly thickness) environments such as handheld devices. The disclosed inductive encoding technology can also be applied in applications requiring high accuracy such as in manufacturing or measurement equipment. The disclosed inductive encoding technology can also be applied in applications with moving metal parts in close proximity such as within a gearbox. The disclosed inductive encoding technology can also be applied in applications in electrically noisy environments where analog voltage measurements (one of the methods used in traditional inductive sensors) are not suitable. In order to overcome the difficulties imposed by size constraints, a low-conductive target with a high permeability may be used in order to increase the inductance of a sense coil. By comparison, a conductive target diminishes the inductance of the sense coil, the low-conductive target with a high permeability allows for a signal swing that is far greater and is less susceptible to interference from surrounding metal, and requires a far smaller sense coil. As taught herein, the target may be formed into different shapes in order to measure not only axial distance, but also rotational or translational position of the target relative to the sense coil or coils. Depending on the embodiment, the target can rotate underneath, over, or between the sensing coils. In one embodiment as illustrated in FIG. 9A and FIG. 9B, the target can rotate underneath or over two sense coils. In another embodiment as illustrated in FIGS. 8A and 8B the target can rotate between two sense coils. As used herein, reference to the target and/or the coils passing relative to each other includes the target rotating under or over one or two sense coils as well as the target rotating between two sense coils, unless specifically called out otherwise.

While various embodiments of the invention have been shown and described herein, it will be clear to those skilled in the art that such embodiments are provided by way of example only. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although some example embodiments may be described herein or in documents incorporated by reference as employing a plurality of units to perform example processes, it is understood that example processes may also be performed by one or a plurality of modules. Additionally, it is understood that the term controller may refer to a hardware device having a processor configured to or programmed to read one or more instructions stored in memory in order to execute the processes described herein in accordance with some embodiments. In some embodiments, the memory is configured to store machine readable instructions and the processor is configured to execute the machine readable instructions to perform one or more processes which are described further below. In some embodiments, multiple different controllers or multiple different types of controllers may be employed in performing one or more processes. In some embodiments, different controllers may be implemented in different portions of a surgical robotic systems.

Some embodiments disclosed herein are implemented on, employ, or are incorporated into a surgical robotic system that includes a camera assembly having at least three articulating degrees of freedom and two or more robotic arms each having at least six articulating degrees of freedom. In some embodiments, the two or more robotic arms can have an additional degree of freedom corresponding to the movement of an associated end-effector (e.g., grasper, manipulator, and the like). In some embodiments, the camera assembly when mounted within a subject (e.g., a patient) can be moved or rotated in a pitch or yaw direction about 180 degrees such that the camera assembly can view rearwardly back towards the insertion site. As such, the camera assembly and the two or more robotic arms can view and operate dexterously forward (e.g., away from the insertion site), to each side, in an upward or downward direction, as well as in the rearward direction to view backwards towards the insertion site. The two or more robotic arms and the camera assembly can also move in the roll, pitch and yaw directions.

In some embodiments, there can be a large number of degrees of freedom in some surgical robotic systems described herein, in comparison to some conventional surgical robotic systems. The large number of degrees of freedom enables movements of a robotic arm assembly and orientations of the robotic arm assembly not possible with some conventional surgical robotic arms and enables movements of a camera of the robotic camera assembly not possible in cameras for some conventional surgical robotic systems. For example, many conventional surgical robotic systems having two robotic arms and fewer degrees of freedom per arm may not be able to change a position or an orientation of a virtual chest of the robotic arm assembly while keeping instrument tips of end effectors of the two robotic arms stationary. As another example, cameras of many conventional surgical robotic systems may only have degrees of freedom associated with movement of a support for the camera extending through a trocar and may have no independent degrees of freedom for movement relative to the support.

Some embodiments described herein provide methods and systems employing multiple different control modes, which may be described as a plurality of control modes herein, for controlling a surgical robotic system before, during or after a robotic arm assembly of the surgical robotic system is disposed within an internal body cavity of a subject. In some embodiments, the robotic arm assembly includes at least two robotic arms, which may be described as a “robotic arm assembly” or “arm assembly” herein. In some embodiments, the robotic arm assembly also includes a camera assembly, which may be also be referred to as a “surgical camera assembly”, or “robotic camera assembly” herein. Each control mode uses sensed movement of one or more hand controllers, and may also use input from one or more foot pedals, to control the robotic arm assembly and/or the camera assembly. A control mode may be changed from a current control mode to a different selected control mode based on operator input (e.g., provided via the one or more hand controllers and/or the one or more foot pedals of the surgical robotic system). In different control modes, the same movements of the hand controllers may result in different motions of the surgical robotic assembly.

When describing the control modes, a reference, an orientation or a direction of view of a “camera assembly” or a “camera” is referring to an orientation or a direction of a component or group of components of the surgical robotic arm assembly that includes one or more cameras or other imaging devices that can collectively change orientation with respect to the robotic arm assembly and provide image data to be displayed. For example, in some embodiments, the one or more cameras or other imaging devices may all be disposed in a same housing whose orientation can be changed relative to a support (e.g., support tube or support shaft) for the camera assembly.

Some embodiments employ a plurality of control modes including an instrument control mode, which may also be referred to as an “instrument mode” herein, as well as one or more additional control modes.

In some embodiments, the additional control modes include a scan mode, which may also be referred to herein as a “scanning mode” or a “survey mode”. In the scan mode, the camera changes orientation to change a direction or an orientation of view in response to movement of one or both of the hand controllers.

In some embodiments, the additional control modes include a view control mode, which may also be referred to as a “view mode”, a “camera control mode”, a “camera mode”, a “framing control mode”, or a “framing mode” herein. In the camera mode/view mode, the surgical robotic system can rotate the camera, can translate a virtual chest of the robotic arm assembly, can pivot the virtual chest of the robotic arm assembly or perform any combination of the aforementioned to change an orientation of a direction of view and a perspective of the camera in response to movement of one or both hand controllers while automatically maintaining a position and an orientation of an instrument tip of each robotic arm stationary.

In some embodiments, the additional control modes include a travel control mode, which may also be referred to as a “travel mode” or an “autotrack mode” herein. In some embodiments, the travel mode is one of multiple tracking modes, in which the surgical robotic arm assembly automatically adjusts so that a view of the camera tracks a position at a midpoint between instrument tips. In the travel mode, the virtual chest of the robotic arm assembly can be translated, the robotic arms and the virtual chest of the robotic arm assembly together can be translated, an orientation of the virtual chest can be changed, an orientation of the camera can be changed, or any combination of the aforementioned, to automatically center the camera view on the instrument tips as the instrument tips are moved in response to movement of one or both hand controllers.

In some embodiments, the additional control modes include a pivot control mode, which may also be referred to as a “pivot mode” herein. In the pivot mode, which is a tracking mode, the orientation of the robotic chest, the camera or both can be changed to automatically center the camera view on the midpoint between the instrument tips as the instrument tips are moved in response to movement of one or both hand controllers.

In some embodiments, the additional control modes include a translate control mode, which may also be referred to as a “translate mode” herein. In the translation mode, which is a tracking mode, the virtual chest of the robotic arm assembly or the virtual chest and the robotic arm assembly can be translated together to automatically center the camera view on the midpoint between the instrument tips while the instrument tips are moved in response to movement of one or both hand controllers.

In some embodiments, the pivot mode, the travel mode and the translate mode may all be referred to as “tracking modes” herein because the view of the camera tracks a midpoint between instrument tips of the robotic arms in these modes.

Some embodiments employ additional features for controlling the robotic arm assembly. For example, some embodiments enable individual control of an elbow bias or an elbow elevation of a right robotic arm and a left robotic arm. Some embodiments employ a graphical user interface that identifies a current control mode of the surgical robotic system. Some embodiments employ a menu feature in which a menu is displayed on the graphical user interface and one or more of the hand controllers can be used to traverse menu options and select menu options.

Some embodiments may be employed with a surgical robotic system. A system for robotic surgery may include a robotic subsystem. The robotic subsystem includes at least a portion, which may also be referred to herein as a robotic arm assembly that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks may be referred to as a surgical robotic module, a surgical robotic module or a robotic arm assembly herein. The surgical robotic module can include multiple different submodules or parts that may be inserted into the trocar separately. The surgical robotic module, or robotic arm assembly can include multiple separate robotic arms that are deployable within the patient. These multiple separate robotic arms may be collectively referred to as a robotic arm assembly herein. Further, a surgical camera assembly can also be deployed along a separate axis. The surgical robotic module, surgical robotic module, or robotic arm assembly may also include the surgical camera assembly. Thus, the surgical robotic module, or robotic arm assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the robotic surgical instruments into a surgical ready state as well as the subsequent removal of the robotic surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other robotic surgical instruments known in the art.

The systems, devices, and methods disclosed herein can be incorporated into and/or used with a robotic surgical device and associated systems disclosed for example in U.S. Pat. No. 10,285,765 and in PCT patent application Serial No. PCT/US2020/39203, and/or with the camera assembly and system disclosed in United States Patent Application Publication No. 2019/0076199, and/or the systems and methods of exchanging surgical tools in an implantable surgical robotic system disclosed in PCT patent application Serial No. PCT/US2021/058820, where the content and teachings of all of the foregoing patents, patent applications and publications are incorporated herein by reference herein in their entirety. The surgical robotic system according to the present disclosure can include a user workstation that includes appropriate sensors and displays, and a robot support system (RSS) for interacting with and supporting the robotic subsystem in some embodiments. The robotic subsystem includes a motor and a surgical robotic module that includes one or more robotic arms and one or more camera assemblies in some embodiments. The robotic arms and camera assembly can form part of a single support axis robotic system, can form part of the SA architecture robotic system, or can have another arrangement. The RSS can provide multiple degrees of freedom such that the robotic module can be maneuvered within the patient into a single position or multiple different positions. In one embodiment, the RSS can be directly mounted to a surgical table or to the floor or ceiling within an operating room. In another embodiment, the RSS can be mounted by various fastening means, including but not limited to, clamps, screws, or a combination thereof. In other embodiments, the structure may be free standing. The RSS can mount a motor assembly that is coupled to the surgical robotic module, which includes the robotic arms and the camera assembly. The motor assembly can include gears, motors, drivetrains, electronics, and the like, for powering the components of the surgical robotic module.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement of the robotic arms with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Patent Application Publication. No. 2018/0221102, the entire contents of which are herein incorporated by reference.

Like numerical identifiers are used throughout the figures to refer to the same elements.

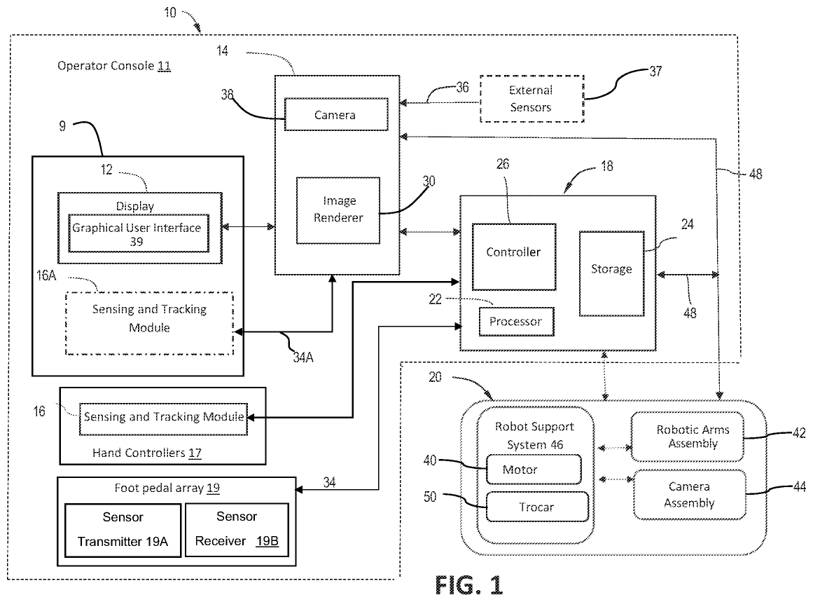

FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display 12, an image computing module 14, which may be a three-dimensional (3D) computing module, hand controllers 17 having a sensing and tracking module 16, and a computing module 18. Additionally, the operator console 11 may include a foot pedal array 19 including a plurality of pedals. The visualization system 9 can include a graphical user interface 39. The graphical user interface 39, the controller 26 or the image renderer 30, or both, may render one or more images or one or more graphical user interface elements on the graphical user interface 39. For example, a pillar box associated with a mode of operating the surgical robotic system 10, or any of the various components of the surgical robotic system 10, can be rendered on the graphical user interface 39. Also live video footage captured by a camera assembly 44 can also be rendered by the controller 26 or the image renderer 30 on the graphical user interface 39.

The operator console 11 can include a visualization system 9 that includes a display 12 which may be any selected type of display for displaying information, images or video generated by the image computing module 14, the computing module 18, and/or the robotic subsystem 20. The display 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display 12 can also include an optional sensing and tracking module 16A. In some embodiments, the display 12 can include an image display for outputting an image from a camera assembly 44 of the robotic subsystem 20.

The hand controllers 17 are configured to sense a movement of the operator's hands and/or arms to manipulate the surgical robotic system 10. The hand controllers 17 can include the sensing and tracking module 16, circuity, and/or other hardware. The sensing and tracking module 16 can include one or more sensors or detectors that sense movements of the operator's hands. In some embodiments, the one or more sensors or detectors that sense movements of the operator's hands are disposed in the hand controllers 17 that are grasped by or engaged by hands of the operator. In some embodiments, the one or more sensors or detectors that sense movements of the operator's hands are coupled to the hands and/or arms of the operator. For example, the sensors of the sensing and tracking module 16 can be coupled to a region of the hand and/or the arm, such as the fingers, the wrist region, the elbow region, and/or the shoulder region. Additional sensors can also be coupled to a head and/or neck region of the operator in some embodiments. In some embodiments, the sensing and tracking module 16 can be external and coupled to the hand controllers 17 via electricity components and/or mounting hardware. In some embodiments, the optional sensor and tracking module 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking module 16 can employ sensors coupled to the torso of the operator or any other body part. In some embodiments, the sensing and tracking module 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking module 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computing module 18 and hence employed by the surgical robotic system 10.

The sensors generate position and/or orientation data indicative of the position and/or orientation of the operator's hands and/or arms. The sensing and tracking module 16 and/or 16A can be utilized to control movement (e.g., changing a position and/or an orientation) of the camera assembly 44 and robotic arm assembly 42 of the robotic subsystem 20. The tracking and position data 34 generated by the sensing and tracking module 16 can be conveyed to the computing module 18 for processing by at least one processor 22.

The computing module 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arms 42 and/or the camera assembly 44. For example, the controller 26 can change a position and/or an orientation of at least a portion of the camera assembly 44, of at least a portion of the robotic arm assembly 42, or both. In some embodiments, the controller 26 can also adjust the pan and tilt of the camera assembly 44 to follow the movement of the operator's head.

The robotic subsystem 20 can include the RSS 46 having a motor 40 and a trocar 50 or trocar mount, the robotic arm assembly 42, and the camera assembly 44. The robotic arm assembly 42 and the camera assembly 44 can form part of a single support axis robot system, such as that disclosed and described in U.S. Pat. No. 10,285,765, or can form part of a SA architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arm assembly 42 and the camera assembly 44 are separately manipulatable, maneuverable, and movable. The robotic subsystem 20, which includes the robotic arm assembly 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arm assembly 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arms 42 and the cameras assembly 44 separately or together. The motor 40 can also provide mechanical power, electrical power, mechanical communication, and electrical communication to the robotic arms 42, the camera assembly 44, and/or other components of the RSS 46 and robotic subsystem 20. The motor 40 can be controlled by the computing module 18. The motor 40 can thus generate signals for controlling one or more motors that in turn can control and drive the robotic arms 42, including for example the position and orientation of each robot joint of each robotic arm, as well as the camera assembly 44. The motor 40 can further provide for a translational or linear degree of freedom that is first utilized to insert and remove each component of the robotic subsystem 20 through a trocar 50. The motor 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar 50 can be used to place at least a portion of the robotic subsystem 20 in an interior cavity of a subject (e.g., a patient) and can withdraw gas and/or fluid from a body cavity. The robotic subsystem 20 can be inserted through the trocar 50 to access and perform an operation in vivo in a body cavity of a patient. In some embodiments, the robotic subsystem 20 can be supported, at least in part, by the trocar 50 or a trocar mount with multiple degrees of freedom such that the robotic arm assembly 42 and the camera assembly 44 can be maneuvered within the patient into a single position or multiple different positions. In some embodiments, the robotic arm assembly 42 and camera assembly 44 can be moved with respect to the trocar 50 or a trocar mount with multiple different degrees of freedom such that the robotic arm assembly 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking module 16, the robotic arm assembly 42, the camera assembly 44, and the like), and for generating control signals in response thereto. The motor 40 can also include a storage element for storing data in some embodiments.

The robotic arm assembly 42 can be controlled to follow the scaled-down movement or motion of the operator's arms and/or hands as sensed by the associated sensors in some embodiments and in some modes of operation. The robotic arm assembly 42 include a first robotic arm including a first end effector at a distal end of the first robotic arm, and a second robotic arm including a second end effector disposed at a distal end of the second robotic arm. In some embodiments, the robotic arm assembly 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control while a virtual chest of the robotic arm assembly 42 may remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure control of movement of individual arms of the robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers 17 grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of stereoscopic cameras in the camera assembly 44 can be configured to provide a user experience that feels natural and comfortable. In some embodiments, the interaxial distance between the stereoscopic cameras can be modified to adjust the depth of the operation site perceived by the operator.

The image or video data 48 generated by the camera assembly 44 can be displayed on the display 12. In embodiments in which the display 12 includes an HMD, the display 12 can include the built-in sensing and tracking module 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking module. In some embodiments, the sensing and tracking module 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensing and tracking module 16A for tracking at least a portion of the operator's head.

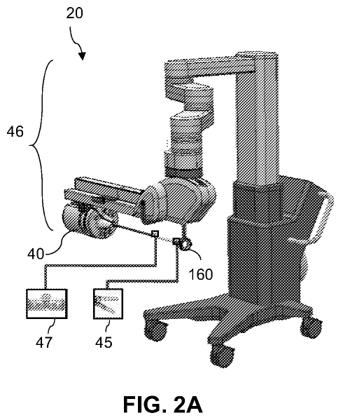

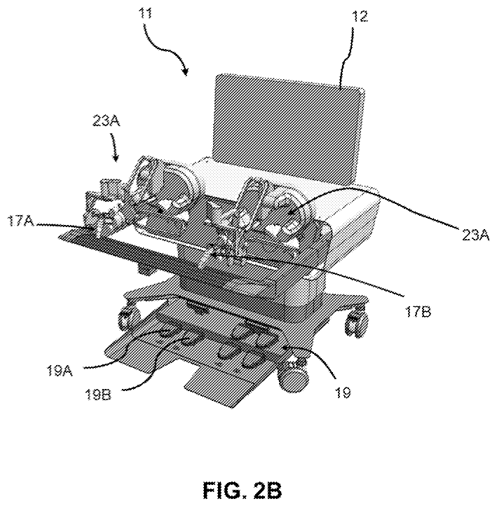

FIG. 2A depicts an example robotic arm assembly 20, which is also referred to herein as a robotic subsystem, of a surgical robotic system 10 incorporated into or mounted onto a mobile patient cart in accordance with some embodiments. In some embodiments, the robotic arm assembly 20 includes the RSS 46, which, in turn includes the motor 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes the display 12, the hand controllers 17, and also includes one or more additional controllers, such as a foot pedal array 19 for control of the robotic arms 42, for control of the camera assembly 44, and for control of other aspects of the system.

FIG. 2B also depicts the left hand controller subsystem 23A and the right hand controller subsystem 23B of the operator console. The left hand controller subsystem 23A can include and support the left hand controller 17A and the right hand controller subsystem 23B can include and support the right hand controller 17B. In some embodiments, the left hand controller subsystem 23A may releasably connect to or engage the left hand controller 17A, and right hand controller subsystem 23B may releasably connect to or engage the right hand controller 17A. In some embodiments, the connections may be both physical and electronic so that the left hand controller subsystem 23A and the right hand controller subsystem 23B may receive signals from the left hand controller 17A and the right hand controller 17B, respectively, including signals that convey inputs received from a user selection on a button or touch input device of the left hand controller 17A or the right hand controller 17B.

In some embodiments, an arm engagement mode is disclosed. The arm engagement mode is an initialization process that guides the user to place the hand controllers 17 into the proper state to match the current state of the robotic arm assembly 42 to prevent unexpected motion from occurring prior to the robotic arm assembly 42 tracking the hand controllers 17. The details of a user involved arm engagement process are discussed in more detail below. The robot pose views 171 and 172 can provide the user with some situational awareness and spatial awareness about the orientation of the robotic arms 42A and 42B.

In some embodiments, the arm engagement process includes the process of engaging the right hand of the user with the right hand controller 202 and engaging the left hand of the user with the left hand controller 201 of the surgical robotic system 10 to ensure that the user places the right hand controller 202 and the left hand controller 201 into a proper state to match the current state of the robotic arm 42A and the robotic arm 42B in such a way that no unexpected motion occurs when the sensing and tracking module 16 begins tracking the right hand controller 202 and the left hand controller 201. This can be accomplished by guiding the user to place their right arm and hand into the correct position and orientation with respect to the right hand controller 202 and guiding the user to place their left arm and hand into the correct position and orientation with respect to the left hand controller 201. The user's right arm and left arm can be referred to as a “matching human right arm” and a “matching human left arm” respectively, and the user's right hand and left hand can be referred to as a “matching human right hand” and a “matching human left hand” respectively.

The process of engaging the user's right hand with the right hand controller 202 also ensures that an instrument 162 (instrument tip, or end effector), for example a grasper coupled to the robotic arm 42B, does not drop a surgical item, such as a suture or tissue, once the user is engaged with the robotic assembly, and begins to control the robotic arm assembly 42. Upon pressing or otherwise manipulating an engagement button or similar input on a hand controller (the right hand controller 202 or the left hand controller 201), the robotic surgical system enters the “intent to engage” mode. This in turn generates a signal to display, on the graphical user interface 150, an engagement guidance cue 197 and an engagement guidance cue 196 such as a matching human engagement ring 154 and an engagement ring 153 and a matching human engagement ring 156 and an engagement ring 155.

Each of the left hand controller subsystem 23A and the right hand controller subsystem 23B may include components that enable a range of motion of the respective left hand controller 17A and right hand controller 17B, so that the left hand controller 17A and right hand controller 17B may be translated or displaced in three dimensions and may additionally move in the roll, pitch, and yaw directions. Additionally, each of the left hand controller subsystem 23A and the right hand controller subsystem 23B may register movement of the respective left hand controller 17A and right hand controller 17B in each of the forgoing directions and may send a signal providing such movement information to a processor (not shown) of the surgical robotic system.

In some embodiments, each of the left hand controller subsystem 23A and the right hand controller subsystem 23B may be configured to receive and connect to or engage different hand controllers (not shown). For example, hand controllers with different configurations of buttons and touch input devices may be provided. Additionally, hand controllers with a different shape may be provided. The hand controllers may be selected for compatibility with a particular surgical robotic system or a particular surgical robotic procedure or selected based upon preference of an operator with respect to the buttons and input devices or with respect to the shape of the hand controller in order to provide greater comfort and ease for the operator.

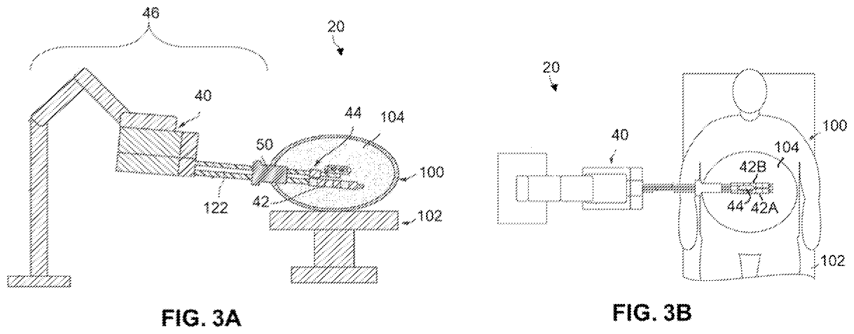

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic arm assembly 42 can be coupled to the motor 40 and at least a portion of the robotic arm assembly 42 can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly 42 may include some portions that remain external to the subject's body in use, references to insertion of the robotic arm assembly 42 and/or the camera assembly 44 into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robotic arm of the robotic arm assembly 42 and then followed by a second robotic arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of the robotic arm assembly is provided in International Patent Application Publications WO 2022/094000 A1 and WO 2021/231402 A1, each of which is incorporated by reference herein in its entirety.

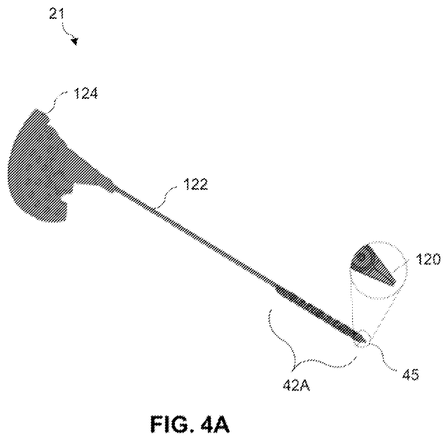

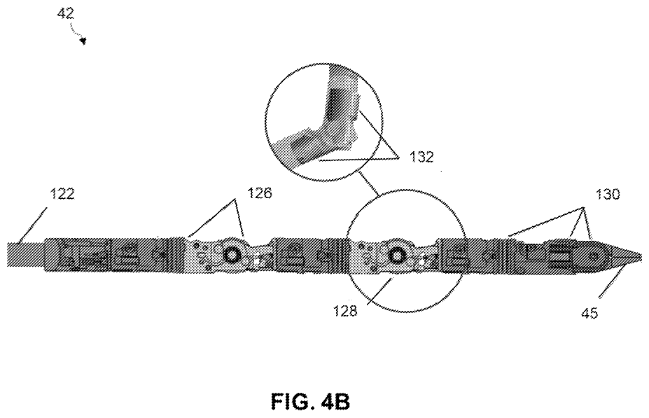

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a virtual shoulder 126, a virtual elbow 128 having position sensor 132 (e.g., inductive sensing coil and oscillator circuit), a virtual wrist 130, and the end-effector 45 in accordance with some embodiments. The virtual shoulder 126, the virtual elbow 128, the virtual wrist 130 can include a series of hinge and rotary joints to provide each arm with positionable, seven degrees of freedom, along with one additional grasping degree of freedom for the end-effector 45 in some embodiments.

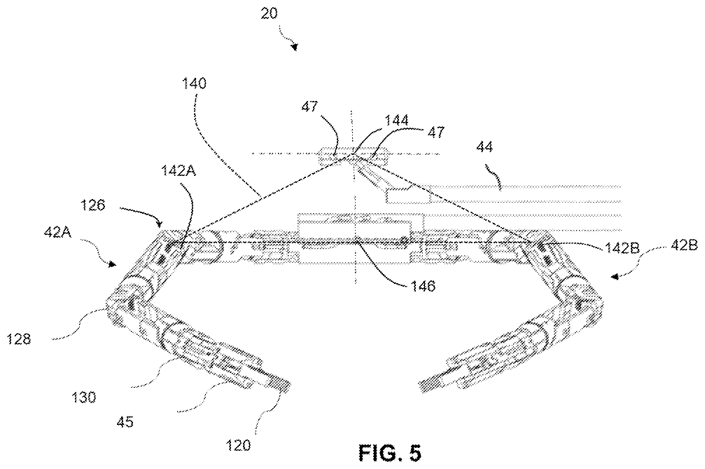

FIG. 5 illustrates a perspective front view of a portion of the robotic subsystem arm assembly 20 configured for insertion into an internal body cavity of a patient. The robotic subsystem arm assembly 20 includes a robotic arm 42A and a robotic arm 42B. The two robotic arms 42A and 42B can define, or at least partially define, a virtual chest 140 of the robotic arm assembly 20 in some embodiments. In some embodiments, the virtual chest 140 (depicted as a triangle with dotted lines) can be defined by a chest plane extending between a first pivot point 142A of a most proximal joint of the robotic arm 42A (e.g., a shoulder joint 126), a second pivot point 142B of a most proximal joint of the robotic arm 42B, and a camera imaging center point 144 of the camera(s) 47. A pivot center 146 of the virtual chest 140 lies in the middle of the virtual chest.

In some embodiments, sensors in one or both of the robotic arm 42A and the robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm. Such sensors are described below in more detail.

In some embodiments, a camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

Hand controllers for a surgical robotic system as described herein can be employed with any of the surgical robotic systems described above or any other suitable surgical robotic system. Further, some embodiments of hand controllers described herein may be employed with semi-robotic endoscopic surgical systems that are only robotic in part.

As explained above, controllers for a surgical robotic system may desirably feature sufficient inputs to provide control of the system, an ergonomic design and “natural” feel in use.

In some embodiments described herein, reference is made to a left hand controller and a corresponding left robotic arm, which may be a first robotic arm, and to a right hand controller and a corresponding right robotic arm, which may be a second robotic arm. In some embodiments, a robotic arm considered a left robotic arm and a robotic arm considered a right robotic arm may change due a configuration of the robotic arms and the camera assembly being adjusted such that the second robotic arm corresponds to a left robotic arm with respect to a view provided by the camera assembly and the first robotic arm corresponds to a right robotic arm with respect to a view provided by the camera assembly. In some embodiments, the surgical robotic system changes which robotic arm is identified as corresponding to the left hand controller and which robotic arm is identified as corresponding to the right hand controller during use. In some embodiments, at least one hand controller includes one or more operator input devices to provide one or more inputs for additional control of a robotic assembly. In some embodiments, the one or more operator input devices receive one or more operators inputs for at least one of: engaging a scanning mode, resetting a camera assembly orientation and position to a align a view of the camera assembly to the instrument tips and to the chest; displaying a menu, traversing a menu or highlighting options or items for selection and selecting an item or option, selecting and adjusting an elbow position, and engaging a clutch associated with an individual hand controller. In some embodiments, additional functions may be accessed via the menu, for example, selecting a level of a grasper force (e.g., high/low), selecting an insertion mode, an extraction mode, or an exchange mode, adjusting a focus, lighting, or a gain, camera cleaning, motion scaling, rotation of camera to enable looking down, etc.

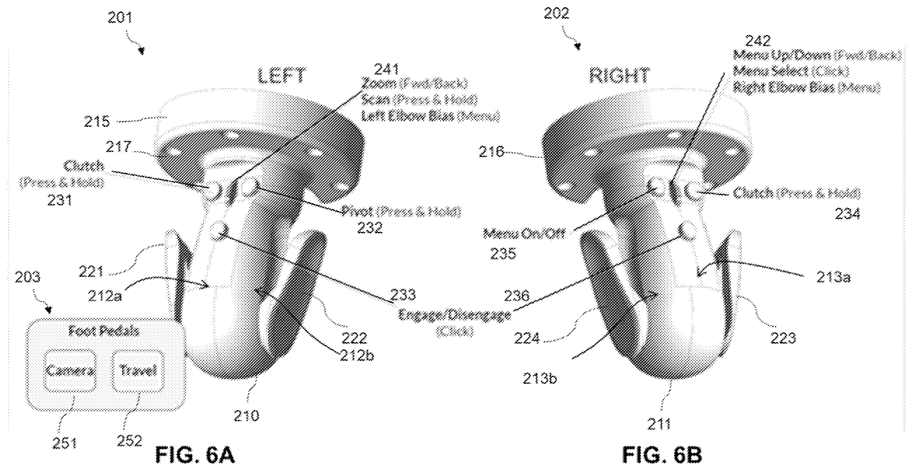

FIG. 6A depicts a left hand controller 201 and FIG. 6B depicts a right hand controller 202 in accordance with some embodiments. The left hand controller 201 and the right hand controller 202 each include a contoured housing 210, 211, respectively. Each contoured housing 210, 211, includes an upper surface 212a, 213a, an inside side surface 212b, 213b adjacent the respective upper surfaces 212a and 213a, an outside side surface (not visible in these views) facing away from the inside side surface 212b, 213b, and a lower surface (not visible in these views) facing away from the upper surface 212a, 213a.

In some embodiments, each hand controller 201, 202 includes a mounting assembly 215, 216, respectively. The mounting assembly 215, 216 may be used to attach, either directly or indirectly, the respective hand controller 201, 202 to a user console of a surgical robotic system. In some embodiments, the mounting assembly 215 defines holes 217, which may be countersunk holes, configured to receive a screw or bolt to connect the left hand controller 201 to a user console.

In some embodiments of the present disclosure, such as that depicted in FIGS. 6A and 6B, the hand controller includes two control levers, three buttons, and one touch input device. As will be explained herein, embodiments may feature other combinations of touch input devices, buttons, and levers, or a subset thereof. The embodiment shown as the left hand controller 201 features a first control lever 221 and a second control lever 222. Similarly, right hand controller 202 includes a first control lever 223 and a second control lever 224. In some embodiments, first control lever 221 is engaged with the second control lever 222 via one or more gears (not shown) so that a user depressing the first control lever 221 causes a reciprocal movement in the second control lever 222 and vice versa. Further description regarding a geared engagement between a first control lever and a second control lever is provided below with respect to FIGS. 11A and 11B. In another embodiment, first control lever 221 and second control lever 222 may be configured to operate independently. In embodiments employing reciprocal movement of the first and second control lever, a hand controller may employ only one signal indicating a deflection of the first lever and the second lever. In embodiments in which the first control lever and second control lever operate independently, a hand controller may employ a first signal indicating a deflection of the first control lever and a second signal indicating a deflection of the second control lever.

In some embodiments, the first control lever 221, 223 and the second control lever 222, 224 may be contoured to receive a thumb and/or finger of a user. In some embodiments, the first control lever 221, 223 extends from or extends beyond the outside side surface of the respective contoured housing 210, 211 the second control lever 222, 224 extends from or extends beyond the inside side surface 213a, 213b of the respective contoured housing. For each hand controller 210, 211, deflection or depression of the first control lever 221, 223, and the second control lever 222, 224, is configured to produce a signal that the surgical robotic system uses as an input to control a tool or an instrument tip (e.g., opening/closing an aperture of graspers/jaws of an instrument tip) at a distal end of a robotic arm of the surgical robotic system. For example, depressing the first control lever and the second control lever may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

In some embodiments, a housing of a hand controller may be contoured. For example, in FIGS. 6A and 6B, the contoured housing 210, 211 includes a rounded shape. In some embodiments, a housing may be shaped to have a contour to match a contour of at least a portion of a thumb of a user's hand. In some embodiments, the contoured housing 210, 211, the first control lever 221, 223, and the second control lever 222, 224, may each be shaped to comfortably and ergonomically receive a respective hand of a user. In some embodiments, a housing of the hand controller, a lever or levers of a hand controller, buttons of a hand controller and/or one or more touch input devices may have shapes and/or positions on the hand controller for fitting different palm sizes and finger lengths.

Left hand controller 201 also includes a first button 231, a second button 232, and a third button 233. Similarly, right hand controller 202 also includes a first button 234, a second button 235 and a third button 236. As will be described herein, each button may provide one or more inputs that may be mapped to a variety of different functions of the surgical robotic device to control the surgical robotic system including a camera assembly and a robotic arm assembly. In an embodiment, input received via the first button 231 of the left hand controller 201 and input received via the first button 234 of the right hand controller 202 may control a clutch feature. For example, by engaging the first button 231, 234 a clutch is activated enabling movement of the respective left hand controller 201 or right hand controller 202, by the operator without causing any movement of a robotic arm assembly (e.g., a first robotic arm, a second robotic arm, and a camera assembly) of the surgical robotic system. When the clutch is activated for a hand controller, movement of the respective right hand controller or left hand controller is not translated to movement of the robotic assembly. In some embodiments, an operator engaging a hand controller input (e.g., tapping or pressing a button) activates the clutch and the operator engaging again (e.g., tapping or pressing the button again) turns off the clutch or exits a clutch mode. In some embodiments, an operator engaging a hand controller input (e.g., tapping or pressing a button and holding the button) activates the clutch and the clutch stays active for as long as the input is active and exits the clutch when the operator is no longer engaging the hand controller input (e.g., releasing the button). Activating the clutch or entering the clutch mode for a hand controller enables the operator to reposition the respective hand controller (e.g., re-position the left controller 201 within the range of motion of the left hand controller 201 and/or re-position the right hand controller 202 within a range of motion of the right hand controller 202) without causing movement of the robotic arm assembly itself.

The second button 232 of the left hand controller 201 may provide an input that controls a pivot function of the surgical robotic device. An operator engaging (e.g., pressing and holding) the second button 232 of the left hand controller 201 may engage a pivot function or a pivot mode that reorients the robotic arm assembly chest to center the camera on the midpoint between the instrument tips. The pivot function can be activated with a brief tap or held down to continuously track the instrument tips as they move, in accordance with some embodiments.

The second button 235 of the right hand controller 202 may provide input for entering a menu mode in which a menu is displayed on the graphical user interface 39 of the surgical robotic system and exiting a menu mode. The operator may activate a menu mode by pressing the second button 235 a first time and disengage the menu function by pressing the second button 235 a second time. The operator may be able to select options within the menu by navigating the menu using the left hand controller and/or the right hand controller when the menu mode is engaged. For example, the first touch input device 242 of the right hand controller 202 may be used to navigate the menu and to select a menu item in some embodiments. While in a menu mode, movement of the robotic arm assembly in response to movement of the left hand controller 201 or the right hand controller 202 may be suspended. The menu mode and the selection of menu options are discussed in more detail below. The third button 233 of the left hand controller and the third button 236 of the right hand controller may provide an input that engages or disengages an instrument control mode of the surgical robotic system in some embodiments. A movement of at least one of the one or more hand controllers when in the instrument mode causes a corresponding movement in a corresponding robotic arm of the robotic assembly. The instrument control mode will be described in more detail below. In some embodiments the surgical robotic arm assembly 10 can employ or provide an instrument control mode 175, which may be described as an “instrument mode” herein. In the instrument mode 175, the surgical robotic system 10 identifies movement (e.g., translation and/or rotation) of each hand controller 201 or 202 and moves (e.g., translates and/or rotates) an instrument tip 120 on a distal end of the corresponding robotic arm 42A or 42B in a corresponding matter. In the instrument control 175 mode, the surgical robotic system 10 may cause an instrument tip 120 to move in a manner directly proportional to movement of a corresponding hand controller 201 or 202.

This may be described as motion including translation and/or rotation of the instrument tip 120 of a robotic arm 42A or 42B being directly controlled by motion of respective hand controller 201 or 202. For example, translating a hand controller 201 or 202 in a direction by an amount causes the corresponding instrument tip 120 for the corresponding robotic arm 42A or 42B to move in a corresponding direction (i.e., in the same direction with respect to a view from the camera assembly 44 displayed to the operator) by a corresponding scaled down amount (e.g., where the scaling is based on the scale of the view from the camera assembly 44 displayed to the operator). As another example, rotating a hand controller 201 or 202 about an axis by an angle causes the corresponding instrument tip 120 for the corresponding robotic arm 42A 42B to rotate by a same angle or by a scaled angle about a corresponding axis (e.g., where the corresponding axis is a same axis with respect to the orientation of the view from the camera assembly 44 displayed to the operator). In the instrument mode 175, operator controls can be used to actuate instruments (e.g., via grasper controls of a hand controller, via foot pedal controls) as well as we as to move or change an orientation of instrument tips 120.

In the instrument mode 175, movement of the hand controllers 201 or 202 does not change a position and does not change an orientation of the camera assembly 44 (e.g., the camera assembly 44 orientation and position may remain fixed) and does not change a position or an orientation of the virtual chest 140. In other words, the instrument mode 175 does not reposition or reorient the camera assembly 44 or the virtual chest 140. The instrument control mode 175 is useful for manipulating the instrument tips 120 within a working area of an internal body cavity that is accessible without moving a virtual chest 140 of the robotic arm assembly 42.

In some embodiments, the operator can enable or disable the instrument control mode 175 via either or both of the hand controllers 201 or 202. In some embodiments, an instrument mode 175 is engaged and disengaged using an input control from a hand controller 201 or 202 (e.g., by pressing a button, such as button 233 in FIG. 6A, or interacting with a touch input device). When the instrument control mode 175 is disengaged, any movement of a hand controller 201 or 202 does not cause any corresponding movement of the associated instrument tip 120. In some embodiments, when the surgical robotic system 10 is in a disengaged state, an information portion of the graphical user interface 150 can indicate that the current state is disengaged. In some embodiments, engaging the clutch causes an information panel of the graphical user interface 150 to identify that the clutch is engaged (e.g., via text, color, or any other graphical indicator).

In some embodiments an operator may put his/her head close to a display such that his/her head is within a certain distance of a display, and the operator can squeeze the paddles as disclosed herein (e.g., as illustrated in FIGS. 6A-8B) to engage an instrument control mode. The operator may pull his/her head away from the display and therefor away from the sensor, that determines how close his/her head is to the display, in order to disengage the instrument control mode.

In some embodiments, the instrument control mode 175 is a default control mode that the surgical robotic system 10 enters when another control mode is exited.

The left hand controller 201 further includes a touch input device 241. Similarly, the right hand controller 202 further includes a touch input device 242. In an embodiment, the touch input device 241, 242 may be a scroll wheel, as shown in FIGS. 6A and 6B. Other touch input devices that may be employed include, but are not limited to, rocker buttons, joy sticks, pointing sticks, touch pads, track balls, track point nubs, etc.

The touch input device 241, 242 may be able to receive input through several different forms of engagement by the operator. For example, where the touch input device 241, 242 is a scroll wheel, the operator may be able to push or click the first touch input device 241, 242, scroll the first touch input device 241, 242 backward or forward, or both.

In some embodiments, scrolling the first touch input device 241 of the left hand controller 241 forward may activate a zoom in function to magnify a view provided by the camera assembly of the surgical robotic system and displayed to the operator, and scrolling backward with first touch input device 241 may provide a zoom out function to reduce the view provided by the camera assembly of the surgical robotic device and displayed to the operator, or vice versa. In embodiments, the zoom function may be mechanical or digital. In some embodiments, the zoom function may be mechanical in part and digital in part (e.g., a mechanical zoom over one zoom range, and a mechanical zoom plus a digital zoom over another zoom range).

In some embodiments, clicking or depressing first touch input device 241 may engage a scan mode of the surgical robotic system. When in a scan mode, a movement of at least one of the left hand controller 201 or the right hand controller 202 causes a corresponding change in an orientation of a camera assembly of the robotic arm assembly 42 without changing a position or orientation of either robotic arm of the surgical robotic system. In another embodiment, pressing and holding the first touch input device 241 may activate the scan mode and releasing the first touch input device 241 may end the scan mode of the surgical robotic system. In some embodiments, releasing the scan mode returns the camera to the orientation it was in upon entering scan mode. In some embodiments, a function may be provided for locking the orientation upon exiting the scan mode (e.g., to change the “horizon” line).

In some embodiments, when in a menu mode and a left elbow menu item is selected, the first touch input device 241 of the left hand controller 201 may be used for selection of a direction and degree of left elbow bias. As used herein, elbow bias refers to the extent by which a virtual elbow of the robotic arm is above or below a neutral or default position.

In some embodiments, when in a menu mode, an operator may be able to select options within the menu by navigating the menu using the left hand controller and/or the right hand controller. For example, when in the menu mode, the touch input device 242 (e.g., scroll wheel) of the right hand controller provide a set of inputs for traversing a displayed menu and selecting an item in a displayed menu. For example, by scrolling forward on touch input device 242 the operator may move up the menu and by scrolling backwards with touch input device 242 the user may move down the menu, or vice versa. In an embodiment, by clicking first touch input device 242 the operator may make a selection within a menu. Use of the touch input device 242 and the menu mode are discussed in more detail below.

In some embodiments, the touch input device 242 of the right hand controller 202 may be used to control right elbow bias when a right elbow bias menu item has been selected.

Functions of various buttons and the touch input devices described above with respect to the left hand controller above may instead be assigned to the right hand controller, and functions of various buttons and the touch input devices described above with respect to the right hand controller may instead be assigned to the left hand controller in some embodiments.

FIG. 6A also shows a schematic depiction 203 of a first foot pedal 251 and second foot pedal 252 for receiving operator input. As shown in FIG. 6A, in some embodiments the first foot pedal 251 engages a camera control mode, also described herein as a view control mode, an image framing control mode, or a camera framing control mode of the surgical robotic system and the second foot pedal 252 engages a travel control mode of the surgical robotic system.

In some embodiments, when the camera control mode is activated e.g., using the first foot pedal 251, movement of the left hand controller 201 and/or the right hand controller 202 by the operator may provide input that is interpreted by the system to control a movement of and an orientation of a camera assembly of the surgical robotic system while keeping positions of instrument tips of robotic arms of the robotic arm assembly constant.

In some embodiments, when the travel control mode is activated e.g., using the second foot pedal 252, the left hand controller 201 and the right hand controller 202 may be used to move the robotic arm assembly of the surgical robotic system in a manner in which distal tips of the robotic arms direct or lead movement of a virtual chest of the robotic arm assembly through an internal body cavity. In the travel control mode, a position and orientation of the camera assembly, of the virtual chest, or of both is automatically adjusted to maintain a view of the camera assembly directed at the tips (e.g., at a point between a tip or tips of a distal end of the first robotic arm and a tip or tips of a distal end of the second robotic arm). This may be described as the camera assembly being pinned to the virtual chest of the robotic arm assembly and automatically following the tips. Further detail regarding the travel control mode is provided below.

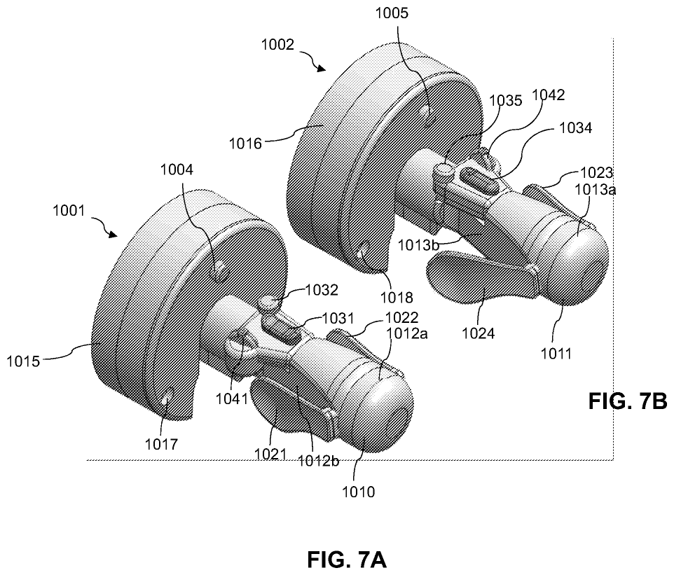

FIGS. 7A and 7B depict another embodiment according to the present disclosure featuring a left hand controller 1001 and a right hand controller 1002. The left hand controller 1001 includes a contoured housing 1010, and the right hand controller 1002 includes a contoured housing 1011. Each contoured housing 1010, 1011, includes an upper surface 1012a, 1013a, an inside side surface 1012b, 1013b adjacent the upper surface, an outside side surface (not visible in these views) facing away from the inside side surface 1012b, 1013b, and a lower surface (not visible in these views) facing away from the upper surface 1012a, 1013a.

Each hand controller 1001, 1002 includes a mounting assembly 1015, 1016, respectively. The mounting assembly 1015, 1016 may be used to attach, either directly or indirectly, each of the respective hand controllers 1001, 1002 to a surgeon console of a surgical robotic system. The mounting assembly 1015 includes an aperture 1017 and the mounting assembly 1016 defines an aperture 1018. The apertures 1017, 1018 may be countersunk apertures, configured to receive a screw or bolt to connect the respective hand controller 1001, 1002 to a surgeon console. The mounting assembly 1015 includes a button 1004 and the mounting assembly 1016 includes a button 1005. The buttons 1004, 1005 provide an input to toggle between insertion and extraction of one or more robotic arm assemblies 42A, 42B as well as the camera assembly 44. For example, the button 1004 can be used to insert or extract a first robotic arm 42A and the button 1005 can be used to insert or extract a second robotic arm 42B.

Each of the left hand controller 1001 and the right hand controller 1002 also includes a first button 1031, 1034, a second button 1032, 1035, a touch input device 1041, 1042 (e.g., a joy stick, or scroll wheel), respectively. In each hand controller 1001, 1002, the first button 1021, 1034, the second button 1032, 1035, and the touch input device 1041, 1042 are disposed on or at an upper surface 1012a, 1013a of the housing 1010, 1011, respectively. In some embodiments, the first button 1021, 1034, the second button 1032, 1035, and the touch input device 1041, 1042 are disposed on or at a portion of the upper surface 1012a, 1013a that projects from the upper surface. For each hand controller 1001, 1002, a lever (not visible in this view) extends from the respective outside side surface (not visible in this view). In some embodiments, a different mechanism may be used for a grasping input on a hand controller. For example, in some embodiments a hand controller may include a least one “pistol trigger” type button that can be pulled back to close and released to open instead of or in addition to a lever or levers.

The left hand controller 1001 includes a first paddle 1021 and a second paddle 1022. Similarly, right hand controller 1002 includes a first paddle 1023 and a second paddle 1024. In some embodiments, the first paddle 1021, 1023 is engaged with the second paddle 1022, 1024 of each hand controller 1001, 1002 via one or more gears (not shown) so that a user depressing the first paddle 1021, 1023 causes a reciprocal movement in the second paddle 1022, 1024 and vice versa, respectively. In another embodiment, the first paddle 1021, 1023 and the second paddle 1022, 1024 of each hand controller may be configured to operate independently. In embodiments employing reciprocal movement of the first and second paddles, the hand controller 1001, 1002 may employ some form of a signal or other indicator indicating a deflection of the first paddle 1021, 1023 and the second paddle 1022, 1024. In embodiments in which the first paddle and second paddle operate independently, the hand controller 1001, 1002 may employ a first signal or other indicator indicating a deflection of the first paddle 1021, 1023 and a second signal or other indicator indicating a deflection of the second paddle 1022, 1024.

In some embodiments, the first paddle 1021, 1023 and the second paddle 1022, 1024 may be contoured to receive a thumb and/or finger of a user. In some embodiments, the first paddle 1021, 1023 extends from or extends beyond the outside side surface of the respective contoured housing 1010, 1011 the second paddle 1022, 1024 extends from or extends beyond the inside side surface 1012b, 1013b of the respective contoured housing. For each hand controller 1010, 1011, deflection or depression of the first paddle 1021, 1023, and the second paddle 1022, 1024, is configured to trigger a signal that the surgical robotic system uses as an input to control a tool or an instrument tip (e.g., opening/closing an aperture of graspers/jaws of an instrument tip) at a distal end of a robotic arm of the surgical robotic system. For example, depressing the first paddle 1021, 1023 and the second paddle 1022, 1024 may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate).

In some embodiments, each of the first paddle 1021, 1023 and the second paddle 1022, 1024 can have a loop to receive a thumb and/or finger of a user, as further described with respect to FIGS. 8A and 8B. In some embodiments, parameters (e.g., length, angle, finger ergonomics, and the like) of each of the first paddle 1021, 1023 and the second paddle 1022, 1024 can be adjusted.

The contoured housing 1010, 1011 may be configured to comfortably and ergonomically mate with a corresponding hand of the operator. The operator may engage with the respective hand controller 1001, 1002 by placing the thumb of the respective hand on the second paddle 1022, 1024, positioning the pointer finger or middle finger of the respective hand on or over the projecting portion of the upper surface 1013a, 1013a on which the first button 1021, 1034, the second button 1032, 1035, and the touch input device 1041, 1042 are disposed, and by positioning at least, the middle finger or ring finger of the respective hand on or over the first paddle 1021, 1024.

Although various example embodiments described herein assign certain functions to certain buttons and to certain touch input devices, one of ordinary skill of the art in view of the present disclosure will appreciate that which functions are ascribed to which buttons and touch input devices may be different in different embodiments. Further, one of ordinary skill of the art in view of the present disclosure will appreciate that additional functions not explicitly described herein may be assigned to some buttons and some touch input devices in some embodiments. In some embodiments, one or more functions may be assigned to a foot pedal of a surgical robotic system that includes one or more hand controllers as described herein.

By way of example, a set of functions that may be controlled by the left hand controller 1001 and the right hand controller 1002 for some embodiments of the present technology will now be described.

For the left hand controller 1001, pressing or pressing and holding the first button 1004 may trigger a signal used to engage an insertion or extraction for a left robotic arm assembly and/or a camera assembly of the surgical robotic system. Pressing or pressing and holding the first button 1031 may trigger a signal used to control a clutch function for the left hand controller of the surgical robotic system. Pressing or pressing and holding the second button 1032 may trigger a signal used to engage or disengage a camera control mode of the surgical robotic system. Scrolling the touch input device 1041 forward may activate a zoom in function to magnify a view provided by the camera assembly of the surgical robotic system and displayed to the operator, and scrolling backward with first touch input device 1041 may provide a zoom out function to reduce the view provided by the camera assembly of the surgical robotic device and displayed to the operator, or vice versa. Scrolling the touch input device 1041 may trigger a signal used to select left elbow bias when an elbow bias function is activated using a menu 1120 (as illustrated in FIG. 26).

For the right hand controller 1002, pressing or pressing and holding the first button 1005 may trigger a signal used to engage an insertion or extraction for a right robotic arm assembly and/or a camera assembly of the surgical robotic system. Pressing or pressing and holding the first button 1034 may trigger a signal used to control a clutch function for the right hand controller of the surgical robotic system. Clicking or depressing the second button 1035 may engage a scan mode of the surgical robotic system. When in a scan mode, a movement of at least one of the left hand controller 1001 or the right hand controller 1002 causes a corresponding change in an orientation of a camera assembly of the robotic assembly without changing a position or orientation of either robotic arm of the surgical robotic system. In another embodiment, pressing and holding the second button 1035 may activate the scan mode and releasing the second button 1035 may end the scan mode of the surgical robotic system. In some embodiments, releasing the scan mode returns the camera to the orientation it was in upon entering the scan mode. In some embodiments, a function may be provided for locking the orientation upon exiting the scan mode (e.g., to change the “horizon” line).

FIGS. 8A and 8B depict another embodiment according to the present disclosure featuring a left hand controller 1001′ and a right hand controller 1002′. Compared with the hand controllers 1001, 1002 in FIGS. 7A and 7B, some buttons of the hand controllers 1001′, 1002′ have the same button type but different functions. For example, the second button 1035′ of the right hand controller 1002′ may trigger a signal used to turn on or turn off a menu. Compared with the hand controllers 1001, 1002 in FIGS. 7A and 7B, some buttons of the hand controllers 1001′, 1002′ may have a different button type and/or different functions. For example, touch input device 1041′ for the left hand controller 1001′ may have a three-way switch button type. Switching or holding the touch input device 1041′ to the center may trigger a signal used to engage or disengage a scan mode of the surgical robotic system. Switching the touch input device 1041′ forward may activate a zoom in function to magnify a view provided by the camera assembly of the surgical robotic system and displayed to the operator, and switching backward with first touch input device 1041′ may provide a zoom out function to reduce the view provided by the camera assembly of the surgical robotic device and displayed to the operator, or vice versa. Switching the touch input device 1035′ upward may trigger a signal used to traverse a menu when the menu is displayed or a menu mode is active. Touch input device 1042′ for the right hand controller 1002′ may have a three-way switch button type. Switching the touch input device 1042′ may trigger a signal used to traverse a menu or highlight a portion of the menu when the menu is displayed or a menu mode is active by pressing the touch input device 1035′. Switching forward on touch input device 1042′ may move up the menu and switching backwards with touch input device 1042′ may move down the menu, or vice versa. Clicking first touch input device 1042′ may trigger a signal used to select a highlighted portion or of the menu or feature on the menu when the menu is displayed. In some embodiments, switching the touch input device 1042′ may trigger a signal used to select right elbow bias when the elbow bias function is activated using the menu. Compared with the hand controllers 1001, 1002 in FIGS. 7A and 7B, the hand controllers 1001′, 1002′ may have the first paddles 1021′, 1023′ and second paddles 1022′, 1024′ to couple to finger loops 1061, 1062, 1063, 1064, respectively. Each finger loop can be a Velcro type. In some embodiments (not illustrated), each finger loop can be a hook type. Deflection or depression of the first paddle 1021′, 1023′, and the second paddle 1022′, 1024′, is configured to trigger a signal to control a tool or an instrument tip (e.g., opening/closing an aperture of graspers/jaws of an instrument tip) at a distal end of a robotic arm of the surgical robotic system. For example, depressing first paddle 1021′, 1023′ and the second paddle 1022′, 1024′ may change an angle of jaws of a grasper at a distal end of the respective robotic arm. In some embodiments, end effectors, tools or instruments are used to pull tissue apart, drive a needle driver, grab an item (e.g., a mesh, suture, needle) or pick up such an item in the body cavity when it is dropped, deliver energy via an electrosurgical unit (ESU) (e.g., to cut or to coagulate). Compared with the hand controllers 1001, 1002 in FIGS. 7A and 7B, first buttons 1031′, 1034′ may have a slider button type. Sliding the first button 1031′, 1034′ may trigger a signal used to control a clutch function for the corresponding hand controller of the surgical robotic system.

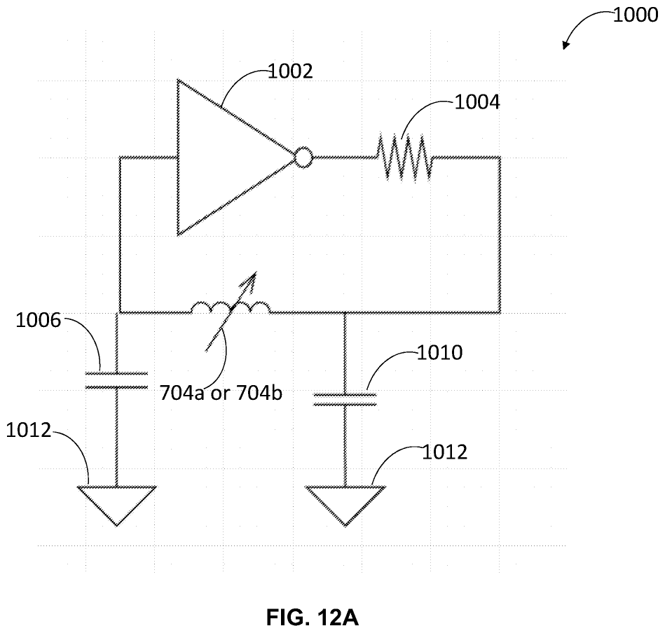

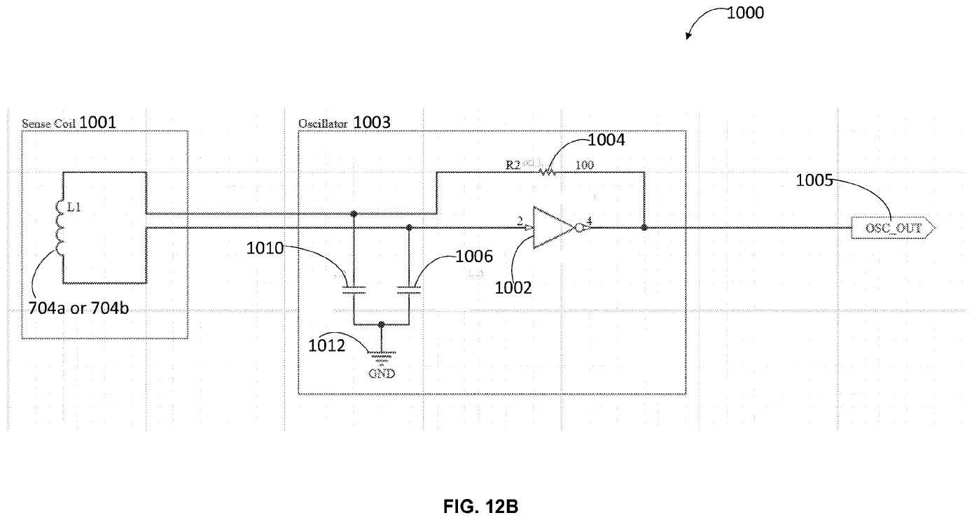

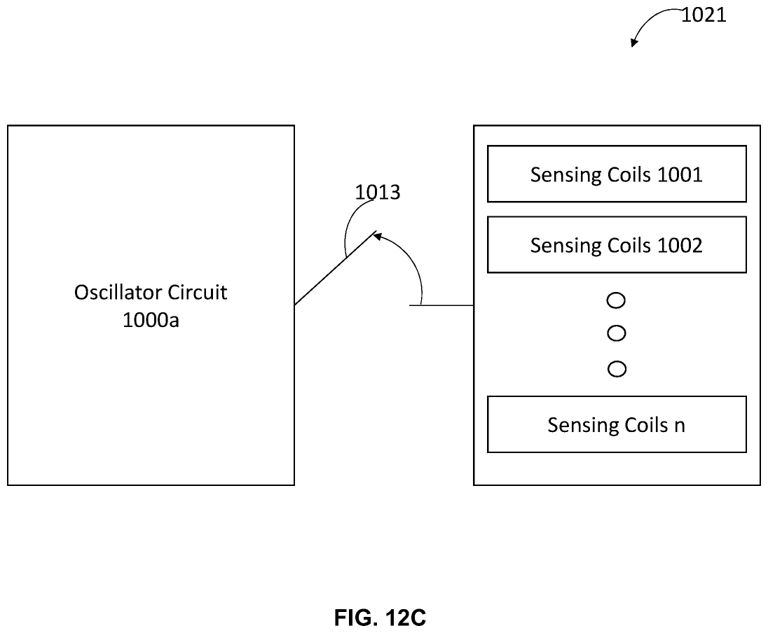

FIG. 9A illustrates a joint 708 of the robotic arm 42A or 42B that includes a position sensor 132. Position sensor 132 can include a target 702 and sensing coils 704a and 704b. The target 702 is in proximity to the sensing coils 704a and 704b so that a change in position of the joint 708 results in a change in resonant frequency of an oscillator circuit that is electrically coupled to the sensing coils 704a and 704b. The position sensor 132 is rotatable with the joint 708 to detect a joint angle or joint position or both. The position sensor 132 is also able to detect an axial distance, or a rotational position, or a translational position, or any combination thereof of the target 702 relative to the sense coils 704a and 704b. As the joint 708 and the position sensor 132 rotates or pivots therewith about axis 706. Depending on the embodiment, the target 702 passes underneath or over, or between the sensing coils 704a or 704b. Depending on how far the joint is rotated, the target 702 passes in relation to one or both of the sensing coil 704a or the sensing coil 704b. In some embodiments, the shape of the target 702 is crescent shape, but it could be a different shape, for example, circular or oval. The sensing coils 704a and 704b can be schematically represented as a variable inductor as illustrated in FIGS. 12A and 12B. As the target 702 passes relative to the sensing coils 704a or 704b, the inductance of the sensing coils 704a or 704b changes because the target 702 can be formed out of a material that has a high permeability, and a low conductivity. In some embodiments, materials that the target is formed of can have differing ratios of conductivity to permeability. The change in inductance of the sensing coils 704a or 704b relative the target 702 corresponds to a change in resonant frequency of an oscillator circuit that is electrically coupled to the sensing coils 704a and 704b. The resonant frequency of the oscillator circuit changes as the target 702 moves relative to the sensing coils 704a or 704b. For example, a first portion of the target 702 is nearly in proximity of the sensing coil 704a but a second portion of the target 702 is completely covering or covered by the sensing coil 704b. In this instance a first unique change in inductance from a baseline value of the sensing coils 704a and 704b can be generated. Accordingly, for each position of the target 702 relative to the sensing coils 704a and 704b, there is a corresponding inductance change and therefore a corresponding change in the resonant frequency of the oscillator circuit that results in a signal that can be measured as joint rotates, and in turn, the position sensor 132 rotates. In some embodiments, one half of the joint is fixed relative the rotation of the other half of the joint. In some embodiments, both halves of the joint move simultaneously.

For each inductance value there is a corresponding resonant frequency that the oscillator circuit operates at. As a result there is a one-to-one, or injective, mapping of each position of the position sensor 132 to an encoder value associated with the inductance measured by an oscillatory circuit. As joint rotates so does the position sensor 132 rotate. As shown in FIG. 9B a third potion of the target 702 is covered by, or is underneath, the sensing coil 704a, but a fourth portion is nearly in proximity of the sensing coil 704b. In this instance a second unique change in inductance from a baseline value of the sensing coils 704a and 704b can be generated, that is different from the first unique change in inductance from the baseline value. A corresponding resonant frequency is generated by the oscillatory circuit which is then encoded into a value that corresponds to a position of the robotic join, and, in turn the position sensor 132.

The shape and arrangement of the sensing coils 704a and 704b can provide precise positioning information of the positioning sensor 132 at all times. This allows for a signal swing that is far greater, is less susceptible to surrounding metal, and requires a far smaller sense coil. The embodiments disclosed herein take advantage of a low-conductive and high permeability target that may be formed into different shapes in order to measure axial distance, as well as rotational or translational position of the target relative to the sense coil or coils.

By making use of a target of varying geometries, the positioning sensor 132 can determine translational or rotational position or a first portion of a robotic joint relative to a second portion of a robotic joint. That is, as the target passes over or under the coil or coils, the inductance increases in proportion to the area of the coil or coils that is covered with the target. In a monotonically increasing target design, this means that each inductance value corresponds to a specific point along the target and therefore a given angle or position. From this foundation, it follows that a desired target design is such that at the “maximum” signal, the target eclipses the sense coil in its entirety and, at the “minimum” signal, the terminal end of the target ends just off of an edge of the sense coil. The design can be further improved by implementing a differential measurement at the joint of interest by placing two sense coils at the joint and modifying the target to have radial symmetry about its midpoint. A total arc of the target is determined by the equation (2*coil_offset_angle)−coil_arc/2. The coil_offset_angle is an angle 710 between the centers of the two sense coils and is equal to the maximum range of motion of the joint (up to a ceiling of 180 degrees-coil_arc/2), whereas “coil_arc” refers to the angle swept by the sense coil itself. This is demonstrated in FIGS. 7A and 9B where we can see a differentially sensed joint with two coils, 704a and 704b and a target 702 designed per the equation above. In FIGS. 7A and 9B, the joint depicted has a range of motion of about 140 degrees, which therefore indicates that the angle between the radial centerlines of coils 704a and 704b i.e. coil_offset_angle, to be 140 degrees as well. With these dimensions defined and knowing that the coils themselves are 40 degrees wide, i.e. coil_arc, we determine that in this embodiment the optimal arc of the target 702 is (2*(140))−(40)/2=260 degrees. Those skilled in the art will appreciate that as taught herein the optimal arc of the target is not limited to 260 degrees. Rather, the above calculation is provided as merely one example to help facilitate explanation of the subject disclosure.

This change in inductance is measured indirectly through measuring the frequency of a modified Pierce oscillator circuit with the inductor taking the place of the crystal. The changing inductance of the coil changes the frequency of the oscillation and the processor is able to determine this. More specifically, it is measuring the period (1/frequency) of the signal by measuring the time between rising edges of the signal. In order to reduce the computational burden of measuring such a fast signal, the process makes use of two timers, where the first is used to divide the frequency into a manageable range (and improving the resolution by averaging over many cycles) and the second timer measures the frequency of this divided signal. This process is detailed in FIG. 17.

With respect to data integrity, while there isn't the concern of communication corruption that occurs when using a communication bus, it is still possible for sources of noise to influence and degrade the measurement. While the averaging process of FIG. 17 demonstrates strong immunity to stray noise, use of a differential measurement or additional shielding (part of which is provided by the materials used within the sensor design) may be pursued for greater immunity.

Additional potential use cases include detection of a user action, measuring user inputs in a constrained space (e.g. the Surgeon Console left or right hand controllers 201 or 202), large scale joint sensing on the Patient cart, and force measurement if coupled with a compliant structure.

FIG. 8A is a side view of an example position sensor with a target, coil sensors, and background in a robotic joint, in accordance with some embodiments. As shown in FIG. 8A the position sensor 132 includes the target 702 a portion of which is in the vicinity of the sensing coil 704a and another portion of the target 702 that is completely covered by or underneath sensing coil 704b. In addition, a circuit board 802 is included which can be included to increase the range of the signal produced by the position sensor 132. FIG. 8B is a top down view of the position sensor 132 with the target 702, the sensing coils 704a and 704b, and the circuit board 802 in the robotic arm. FIG. 8B shows a sensor stack comprising two coils separated by a distance of about 0.5 mm between which the target 702 travels between as the robotic arm 42A or 42B rotates.

FIG. 9 is an example shape of the target 702 as well as the sensor coils 704a and 704b used in the position sensor 132, in accordance with some embodiments. In some embodiments, the target 702 can be ring shaped with varying width or thickness or both around the circumference of the ring. The target 702 can be formed of a material that has a high permeability while having a low conductivity such as shielding films that may be punched into a pattern that is useful as an encoder target. The sensing coils 704a and 704b can have a high conductivity and low permeability.

FIG. 12A is an example schematic of a sense coil and oscillator circuit that is electrically coupled to the position sensor 132 to output an encoder signal associated with a position of a robotic joint, in accordance with some embodiments. Circuit 1000 includes the sensing coil 704a or the sensing coil 704b which can be represented as a variable inductor. In some embodiments, the variable inductor is a single coil. The variable inductor can be shaped to maximize the signal it receives while minimizing the space it occupies within the robotic joint. However, in some embodiments the variable inductor could represent multiple coils connected in series that together perform a function similar to that in a single coil. In the instant disclosure there can be multiple independent copies of the oscillator circuit in FIG. 12A in order to obtain measurements of the target. The circuit further includes an oscillatory circuit formed by capacitors 1006 and 1010, as well as inverter 1002 and resistor 1004 and a ground 1012. In some embodiments, the variable inductor represents a single coil. In such embodiments the coil can be shaped to maximize the signal it receives while minimizing the space it occupies in a space-constrained design. In some embodiments, the variable inductor represents multiple coils in series that together perform a similar function. In such embodiments, circuit 1000 can be repeated to support the multiple coils.

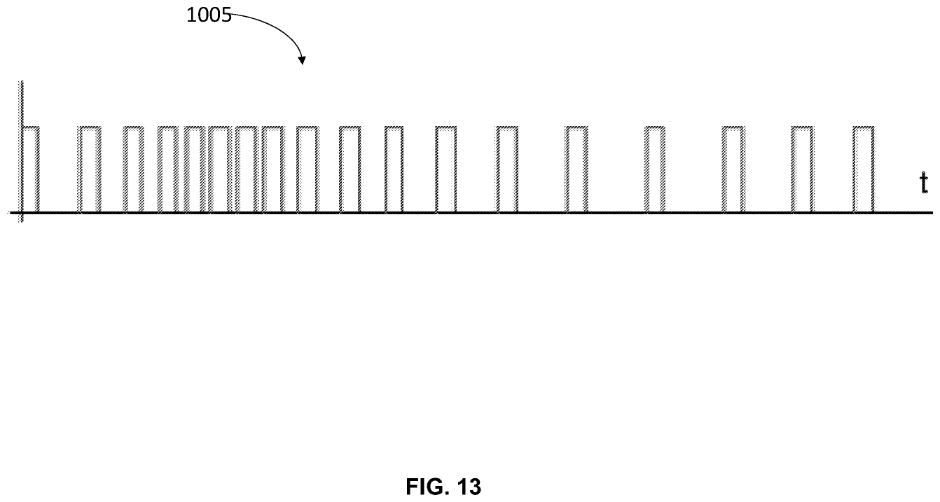

FIG. 12B illustrates the output of circuit 1000 which outputs an output waveform denoted by oscillator circuit output (OSC_OUT) 1005. OSC_OUT 1005 can be a square wave such as the one shown in FIG. 13 in which a signal corresponding to the sensed inductance is encoded in the frequency of the waveform output on OSC_OUT 1005. In some embodiments, there can be a plurality of sensing coils each of which is connected to an individual oscillator circuit. In other embodiments, a first subset of the plurality of sensing coils can be connected to a single oscillator circuit, and a second subset of the plurality of sensing coils can be connected to another oscillator circuit. Further still, the second subset of the plurality of sensing coils can be connected to the single oscillator circuit via a switching mechanism. The switching mechanism can switch between being connected to the first subset of the plurality of sensing coils and the second subset of sensing coils.

FIG. 12C is an example switching circuit 1021 connecting oscillator circuit 1000a to sensing coils 1, 2, . . . n via switch 1013, where n can represent any natural number. That is in certain embodiments, the oscillator circuit 1000a can connect to any number of the sensing coils 1, 2, . . . n, or can connect to any subset of the sensing coils 1, 2, . . . , n.

In some embodiments, the shape of the target and the positional relationships of two or more of the sensing coils 1, 2, . . . , n relative to the joint's range of motion can provide signals from their respective oscillator circuit(s) such that the signals change in approximately opposing manners with joint displacement. There can be computing unit(s) communicatively coupled to the oscillator circuit(s), and the computing unit(s) can combine the signals by measuring a difference of the signals and using the resulting difference in order to determine the displacement of the joint, thereby providing resistance to disturbances inducing a similar change in the signals due to noise or nonidealities in the joint or drift in the oscillators due to temperature. In some embodiments, the signals produced by each of the one or more sensing coils 1, 2, . . . , n can be designed in such a way that subsets of the signals when added together result in a value that is related to the combination of the subsets of the signals. This is referred do as a differential measurement. Taking a difference between the subsets of the signals acts to cancel out disturbances that affect multiple similarly constructed signals. The disturbances experienced by similarly constructed signals can be referred to as common-mode disturbances. Summing the subsets of the signals can preserve or even amplify the true, or actual signals of the subset of the signals.

As an example, consider a first signal generated by a first coil that is represented by the variable y₁ which is based on, or related to, another signal x in addition to some disturbance d. The signal x is representative of the position of the joint along the direction in which the movement of the joint is being sensed. There is a corresponding signal y that is representative of the period of the oscillator circuit, and as noted above the variable d can be any disturbance that changes the period at which the oscillator circuit operates at by a similar amount regardless of where the coil is positioned relative to the target.

That is the signal produced by the first coil can be y₁=x+d. Consider a second signal generated by a second coil that is represented by the variable y₂ which is based on, related to, another signal −x in addition to some disturbance d. That is the signal produced by the second coil can be y₁=−x+d. The processor 22 can calculate a differential measurement between y₁ and y₂ such that m=y₁−y₂=y₁=x+d−(y₂=−x+d)=2x. Because the resulting differential measurement is equal to 2x, and the signal x is known the differential measurement is amplified by a multiple of 2. Temperature and far-field electromagnetic noise sources both tend to generate disturbances in the same direction. As a result, in some embodiments the targets disclosed herein can be designed in such a way that the signal generated by the first coil and the signal generated by the second coil are in opposition to one another. The disclosed targets can have a particular shape such that the signal generated by the first coil and the signal generated by the second signal change in opposition to one another, in which a given change in angle or distance provides resistance to the disturbances.

In some embodiments, the shape of the target and a positional relationship of two or more coils relative to the joint's range of motion provide continuous and periodic signals from their respective oscillator circuit(s) such that the signals, being continuous and periodic, no longer have a 1 to 1 mapping of signal to joint displacement and there can be ambiguity within the period of such signals. A continuous and periodic signal can attain any given value in its range at two or more points within its period. Some additional information is then required to disambiguate (an intra-period ambiguity) the position of the joint when it is near these values. For example, one skilled in the art will that as used herein the intra-period ambiguity refers to the following. A continuous and periodic signal by definition attains any given value in its range at two or more points within its period. If a signal is continuous, it cannot “jump” suddenly between values, it must behave smoothly without skipping points between two values. If a signal is periodic, then after some amount of angular travel (or distance or time or whatever the x-axis is) aka the “period”, the output signal (y-axis) repeats. If a signal is both continuous and periodic, then by definition the output signal has to end each period where it starts the next one. So signals of this nature have a what goes up must come down nature to them within the period, namely that any particular value as the signal goes up must be repeated on the way back down before the signal restarts again in the next period. So for any given value, there are at least two “angles” that value occurs within a period. If a given sensor measures that value, it is not obvious which of the two possible “angles” the joint is at, unless you have additional sensors set up to resolve the intra-period ambiguity.

As taught herein, the shape of the target 702 and the positional relationship of the coils 704 allows the value of one signal to be used to differentiate between the repeated values of a second signal. In this way, each signal may have two options of joint position for a given sensed value, but the other signal can narrow it down to one, the actual joint position.

The computing unit(s) can use the relative positional relationships between coils to determine an expected phase offset of the signals generated by each oscillator circuit. The computing unit(s) can then resolve an intra-period ambiguity of any one signal corresponding to a first coil, using an additional signal corresponding to a second coil, in order to determine the displacement of the joint within a period. This is commonly referred to as quadrature encoding. The target shape and the positional relationship of the coils allows the value of one signal to be used to differentiate between the repeated values of a second signal. In this way, each signal generated by a coil can have two values that could potentially correspond to the joint position. However, a signal generated by another coil can be used to narrow down which of the two values corresponds to the joint position.

In some embodiments, more complex periodic signals can be used with more than two repeated signal values, and additional signals with unique phase offsets can be used to narrow the complex periodic signals to a single possible joint position. By way of example, if two coils are measuring a sinusoidal target, a useful quadrature encoding arrangement of the two coils would be to position them so they read the target with an expected phase difference in their signals of ninety degrees. When a coil's signal is above or below the periodic signal mean value, the processor can uniquely determine which of the two possible points on the target the second coil is reading.

In some embodiments, the computing unit(s) can monitor the number of cycles of the periodic signal that have elapsed, counting whole or partial cycles of joint motion in one direction as positive, and whole or partial cycles of joint motion in the other direction as negative, such that the total displacement of the joint from a starting point can be determined. In some embodiments, the computing unit(s) can receive an input from a user to indicate the starting point as an absolute reference to displacement of the joint.

FIG. 13 is an example output waveform OSC_OUT 1005 produced by the oscillatory circuit 1000, in which the frequency of the oscillation of the output waveform represents the angle/position of the position sensor 132. As each inductance value, and therefore resonant frequency, is unique to each sensed point along the target, the angle or position may be directly mapped from the frequency of the output waveform OSC_OUT 1005. This can be further refined by a calibration step to account for any non-idealities present within the position sensor 132, however this step is not inherently needed by the design. In some embodiments, it may be sufficient for calibration purposes to determine the output waveform OSC_OUT 1005 frequency at the extreme ends of the joint (i.e., maximum and minimum rotation locations of the joint) and interpolate linearly between the two ends. For example, if the output waveform OSC_OUT 1005 has a frequency of 1 MHz with the joint positioned at 0 degrees and the output waveform OSC_OUT 1005 has a frequency of 3 MHz with the point positioned at 90 degrees, it may be inferred that if the output waveform OSC_OUT 1005 has a frequency of 2 MHz, then the joint is positioned at 45 degrees.

FIG. 14 is a prior art depiction of a relationship between a high conductivity and low permeability of a target with a plastic or air background in a position sensor of a robotic arm. For example, the target can have a high conductivity and can be represented by red triangle 1201, and the air can be represented by white triangle 1203. Red triangle 1201 can be a target made of copper, or another high conductivity and low permeability material, that moves relative to a background of air 1202 or some other low conductivity and low permeability material such as a vacuum, or plastics. In some embodiments, the coil is comparable in size with the width of the target, yet small enough relative to the length of the target, so that the inductance of the coil is affected largely by the width of the target at a particular joint position. That is to say that the change in inductance of the coil is directly related to the change in the width of the target as it passes, over, under, or through two coils. For example, the width of the target 702 is not uniform and varies along the length of the target 702. The target 702 has a crescent shape and therefore the width of the target 702 is larger in the middle of the target 702 than the width of the target at the ends of the target 702. As a result, when the middle of the target 702 is within proximity of the coil 704a or coil 704b the inductance of the coil 704a or coil 704b, will be −10 Henrys. And when the ends of the target 702 are within proximity of the coil 704a or coil 704b the inductance of the coil 704a or coil 704b, will be 0 Henrys. The vertical axis in FIG. 14 represents the inductance of a coil and the horizontal axis represents the portion of the target that is within proximity of the coil and hence the position of the joint. The leftmost side of the horizontal axis of FIG. 14 coincides with when the maximum width of the target is within proximity of the coil and the inductance of the coil is −10 Henrys, and the rightmost side of the horizontal axis of FIG. 14 coincides with when the minimum width of the target is within proximity of the coil and the inductance of the coils is 0 Henrys. The total change in inductance=10. As the coil slides along the triangular target, or the target slides along the coil, from left to right, the inductance of the coil increases, and the oscillator frequency decreases. The change is oscillator frequency of the oscillator circuit is measured, which in turn is used to determine the position of the joint.

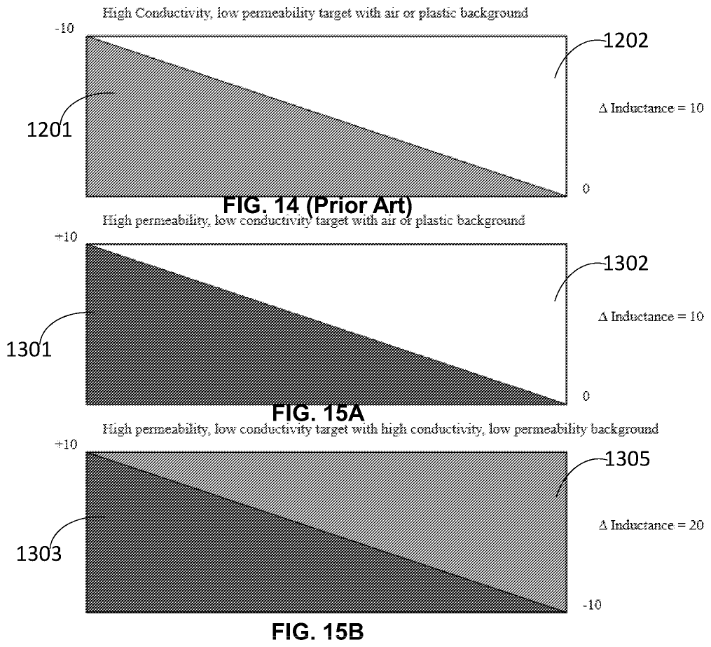

FIG. 15A depicts a relationship between a low conductivity and high permeability of a target with a plastic or air background in the position sensor 132, in accordance with some embodiments. For example, target 702 can have a low conductivity but high permeability material represented as area 1301. The triangular shape of the area 1301 represents the maximum width of the target 702 to it's minimum width. Instead of using a conductive target 9 (See FIG. 14) where eddy currents in a target oppose the magnet field established by the sensing coil, which decreases the inductance compared to the coil being surrounded by a vacuum, or air, or other electromagnetically inert material, FIG. 15A illustrates the behavior of the target 702 formed of a material with a high permeability relative to vacuum or air 1301. FIG. 15A graphically illustrates the concept of the target 702 relative, for example, on a background of air or vacuum or plastic or low permeability and low conductivity material 1302. When the coil 704 is positioned in proximity with the target 702 at its maximum width (left), for example, with respect to the target 702 the maximum width can be the midpoint between the two terminal ends, the inductance of the coil 704 is increased by 10 Henrys. Those skilled in the art will appreciate the maximum width of the target 702 can vary based on the embodiment and shape of the target 702. When the coil 704 is positioned over the minimum width (right), for example, the terminal ends of the target 702, the inductance is at minimum 0 Henrys. Those skilled in the art will appreciate the minimum width of the target 702 can vary based on the embodiment and shape of the target 702. As such, the inductance change is 10 Henrys from maximum width to minimum width and vice versa with reverse trend as compared to the prior art of FIG. 14. As the coil 704 moves left to right relative to the target 702 or vice versa, the inductance decreases, and the oscillator frequency increases.

FIG. 15B depicts the relationship between a high permeability, low conductivity target with high conductivity, low permeability background. FIG. 15B depicts the combination of the effects of a high conductivity, low permeability target of FIG. 14 and a low conductivity, high permeability target of FIG. 15A. Since the inductance in FIG. 15A decreases as the coil 704 moves from the maximum width of the target 702 to the minimum width of the target 702, along the decreasing width of the target, and the high conductivity, low permeability material target used in the FIG. 14 decreases inductance as it's width increases (the opposite effect), we can combine both targets to stack their effects. Instead of a neutral background like air, putting the high permeability, low conductivity target represented by the areas 1301 and 1303 relative to, for example, on a background of a low permeability material 1305. As the width of one decreases the width of the other increases. As a result, twice the effect is a consequence, with the coil 704 having a relative inductance of +10 Henrys when over the maximum width of the target 704 formed from a high-permeability material, and a relative inductance of −10 Henrys when over the maximum width of high-conductivity material, for a total change in inductance of 20 Henrys without requiring a larger coil.

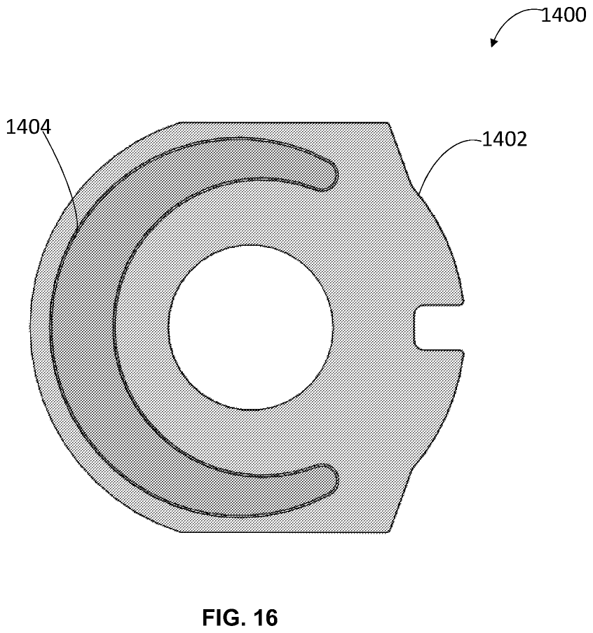

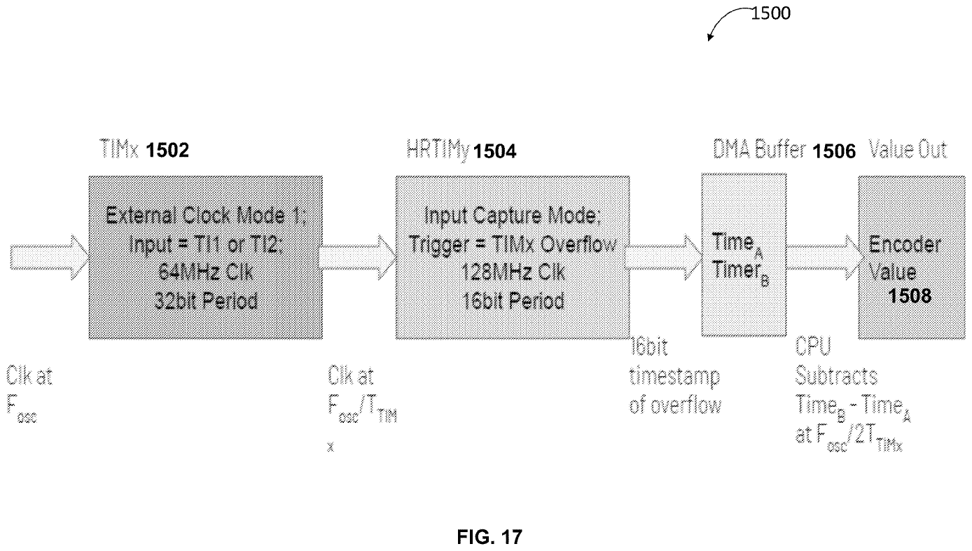

FIG. 16 depicts a target 1400 made of two materials one of which can have a low conductivity and high permeability while the other material has a high conductivity and low permeability, in accordance with some embodiments. Target 1400 can comprise of a material 1402 that has a low conductivity and a high permeability and a material 1404 that has a high conductivity and a low permeability. In some embodiments, the material 1402 can have a high conductivity and a low permeability and the material 1404 can have a low conductivity and a high permeability. In some embodiments, the material 1402 can be a backer material that shields a non-sensing side of the sensing coil, and can help improve the sensing capabilities of the sensing coil.

Target 1400 is a rotational version of a target as taught herein with properties like a in target graphically depicted as 1303 in FIG. 15B. Target 1400 is comprised of two sections. A first section 1404 which has properties like those in target 1303 or target 1301, and a second section 1402 which has properties like those of target 1305 or target 1201. In some embodiments, the first section 1404 and the second section 1402 could be made of the opposite kinds of material. For example, the second section 1402 can have properties like those graphically represented by the area 1303 or graphically represented by the area 1301, and the first section 1404 can have properties like those graphically represented by the area 1305 or graphically represented by the area 1201.

FIG. 17 is a flow diagram 1500 for generating an encoder value 1508 associated with a position of a robotic arm based on the position sensor 132, in accordance with some embodiments. At block 1502, the output waveform OSC_OUT 1005 is divided down to a lower frequency, which is easier to measure, and, in the process, improves the resolution by averaging over many cycles. At block 1504, the moment of each rising edge of the divided signal is recorded in a manner akin to a stopwatch. At block 1506, the frequency of the divided signal is calculated by taking the difference between the recorded moments.

In some embodiments, the OSC_OUT 1005 can be divided down to a lower frequency using two different timers. The first timer divides the frequency of OSC_OUT 1005 into a more manageable range of frequencies which has the natural consequence of improving the resolution of the frequency. More specifically the first timer does not measure the time at the beginning of each cycle or period of OSC_OUT 1005. That is when a rising edge of OSC_OUT 1005 occurs the first timer does not measure the time between consecutive rising edges of OSC_OUT 1005, but rather measures OSC_OUT 1005 at some multiple of each cycle or period and averages the amplitude of OSC_OUT 1005. This can be referred to as dividing the frequency of OSC_OUT 1005. Instead of OSC_OUT 1005 being measured each cycle or period, it is measured at a less frequent time step, but the values of the amplitude of the OSC_OUT 1005 are averaged between the periods in which the frequency of OSC_OUT 1005 are being measured thereby preventing data from being discarded. Because the frequency of OSC_OUT 1005 is divided in such a way that it is not being measured at each rising edge of the signal, a second timer is required to measure the frequency OSC_OUT 1005 at the less frequent time step. The combination of the first timer and the second timer have the effect of retaining the measurement resolution of the frequency of OSC_OUT 1005 without having to measure the frequency of OSC_OUT 1005 at each rising edge of OSC_OUT 1005 thereby decreasing the computational burden on the processor 22.

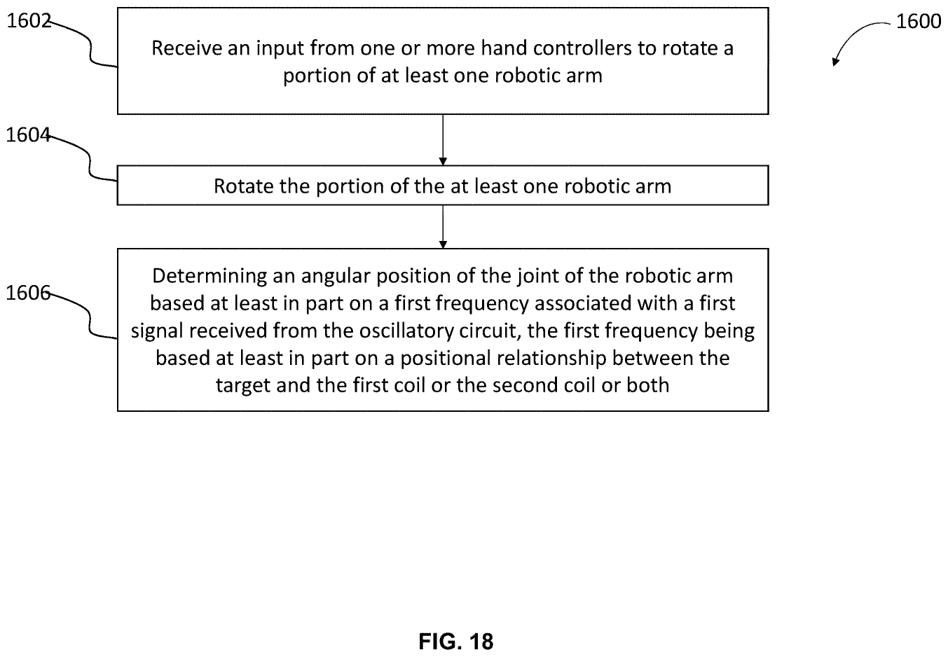

FIG. 18 is an example flowchart 1600 corresponding to determining a position of a robotic arm 42A or 42B using an inductive sensing method, in accordance with some embodiment. For instance, in one embodiment the target 702 sweeps through the area underneath or above the sensing coil 704a and the sensing coil 704b which in turn causes a change in a magnetic field within the sensing coils thereby changing the inductance of the sensing coil 704a and the sensing coil 704b. At block 1602, the robotic subsystem 20 can receive an input from one of the left hand controller 201 or the right hand controller 202 to rotate at least a portion of at least one robotic arm. At block 1604, the robotic subsystem 20 can rotate the position of the portion of the at least one robotic arm, and at block 1606, the robotic subsystem can determine an angular position of a joint of the robotic arm based at least in part on a first frequency associated with a first signal received from an oscillatory circuit, for example, the output waveform OSC_OUT 1005, the first frequency being based at least in part on a positional relationship between the target and the first coil or the second coil or both. For instance, the output waveform OSC_OUT 1005 can be based at least in part on the positional relationship between the target 702 the sensing coil 704a and the sensing coil 704b as shown in FIG. 8A.

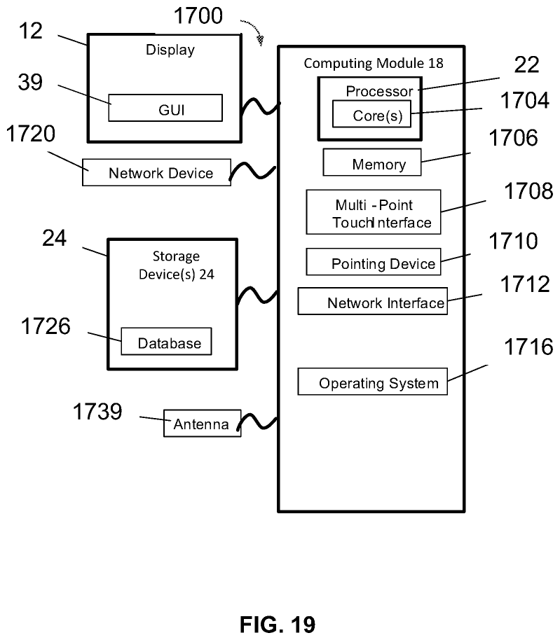

FIG. 19 schematically depicts an example network environment 1700 that the surgical robotic system can be connected to in accordance with some embodiments. Computing module 18 can be used to perform one or more steps of the methods provided by example embodiments. The computing module 18 includes one or more non-transitory computer-readable media for storing one or more computer-executable instructions or software for implementing example embodiments. The non-transitory computer-readable media can include, but are not limited to, one or more types of hardware memory, non-transitory tangible media (for example, one or more magnetic storage disks, one or more optical disks, one or more USB flashdrives), and the like. For example, memory 1706 included in the computing module 18 can store computer-readable and computer-executable instructions or software for implementing example embodiments, for example the steps of method 1600. The computing module 18 also includes the processor 22 and associated core 1704, for executing computer-readable and computer-executable instructions or software stored in the memory 1706 and other programs for controlling system hardware. The processor 22 can be a single core processor or multiple core (1704) processor.

Memory 1706 can include a computer system memory or random access memory, such as DRAM, SRAM, EDO RAM, and the like. The memory 1706 can include other types of memory as well, or combinations thereof. A user can interact with the computing module 18 through the display 12, such as a touch screen display or computer monitor, which can display the graphical user interface (GUI) 39. The display 12 can also display other aspects, transducers and/or information or data associated with example embodiments. The computing module 18 can include other I/O devices for receiving input from a user, for example, a keyboard or any suitable multi-point touch interface 1708, a pointing device 1710 (e.g., a pen, stylus, mouse, or trackpad). The keyboard 1708 and the pointing device 1710 can be coupled to the visual display device 12. The computing module 18 can include other suitable conventional I/O peripherals.

The computing module 18 can also include one or more storage devices 24, such as a hard-drive, CD-ROM, or other computer readable media, for storing data and computer-readable instructions, applications, and/or software that implements example operations/steps of the surgical robotic system 10 as described herein, or portions thereof, which can be executed to generate GUI 39 on display 12. Example storage devices 24 can also store one or more databases for storing any suitable information required to implement example embodiments. The databases can be updated by a user or automatically at any suitable time to add, delete or update one or more items in the databases. Example storage device 24 can store one or more databases 1726 for storing provisioned data, and other data/information used to implement example embodiments of the systems and methods described herein.

The computing module 18 can include a network interface 1712 configured to interface via one or more network devices 1720 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 1712 can include a built-in network adapter, network interface card, PCMCIA network card, card bus network adapter, wireless network adapter, USB network adapter, modem or any other device suitable for interfacing the computing module 18 to any type of network capable of communication and performing the operations described herein. Moreover, the computing module 18 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing module 18 can run any operating system 1716, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In some embodiments, the operating system 1716 can be run in native mode or emulated mode. In some embodiments, the operating system 1716 can be run on one or more cloud machine instances.

The computing module 18 can also include an antenna 1730, where the antenna 1730 can transmit wireless transmissions a radio frequency (RF) front end and receive wireless transmissions from the RF front end.

1. A system for determining an angular position of a rotary join, the system comprising:
a coil coupled to a first portion of the rotary joint at a first location;
a target having high permeability and low conductivity properties, coupled to a second portion of the rotary joint;
an oscillator circuit communicatively coupled to the coil; and
a computing unit communicatively coupled to the oscillator circuit, the computing unit comprising a processor configured to or programmed to:
determine an position of the rotary joint based at least in part on a frequency associated with a signal received from the oscillatory circuit, the frequency being based at least in part on a positional relationship between the target and the coil.

2. The system of claim 1, further comprising a second coil coupled to the joint at a second location.

3. A system for determining the rotational or translational position of a joint, the system comprising:
a first coil coupled to a first portion of the joint at a first location;
a target having high permeability and low conductivity properties, coupled to a second portion of the joint; and
an oscillator circuit communicatively coupled to the first coil which generates a variable frequency that is correlated with the position as an output.

4. The system of claim 3, wherein the joint has multiple degrees of freedom.

5. The system of claim 3, further comprising one or more second coils coupled to the joint at a second location separate from the first location, and wherein the one or more second coils are communicatively coupled to the oscillator circuit.

6. The system of claim 5, wherein a subset of the one or more second coils are communicatively coupled to the same oscillator circuit through a switching mechanism.

7. The system of claim 3, wherein the target is composed of two or more materials with differing ratios of conductivity to permeability.

8. The system of claim 3, further comprising a backer material that shields a non-sensing side of the coil.

9. The system of claim 3, further comprising:
a computing unit communicatively coupled to the oscillator circuit, the computing unit comprising a processor configured to or programmed to:
determine an angular position of the joint based at least in part on a first frequency associated with a first signal received from the oscillator circuit, the first frequency being based at least in part on a positional relationship between the target and the first coil.

10. A system for determining displacement of a joint of a mechanism, the system comprising:
a first coil coupled to a first portion of a joint at a first location;
a target having high permeability and low conductivity properties, coupled to a second portion of the joint;
an oscillator circuit communicatively coupled to the first coil; and
a computing unit communicatively coupled to the oscillator circuit, the computing unit comprising a processor configured to or programmed to:
determine displacement of the joint of the mechanism based at least in part on a first frequency associated with a first signal received from the oscillator circuit, the first frequency being based at least in part on a positional relationship between the target and the first coil.

11. The system of claim 10, wherein the target abuts a background material having a low permeability and high conductivity.

12. The system of claim 10, wherein the target is made from a low permeability and high conductivity material; and wherein the target abuts a background material having high permeability and low conductivity.

13. The system of claim 10, further comprising:
one or more additional coils coupled to the second portion of the joint;
one or more additional oscillator circuits communicatively coupled to a respective one of the coils; and
the computing unit communicatively coupled to the one or more additional oscillator circuit(s):
determines the displacement of the joint of the mechanism based at least in part on a set of frequencies associated with the signals received from the oscillatory circuits, the set of frequencies being based at least in part on positional relationships between the target and the one or more additional coils.

14. The system of claim 13, wherein the shape of the target and the positional relationships of two or more coils relative to the joint's range of motion provide signals from their respective oscillator circuit(s) such that:
the signals change in approximately the same manner with joint displacement; and
the computing unit(s) removes outliers and averages the signals and uses the results to:
determine the displacement of the joint of the mechanism, thereby providing redundancy from failure, and resistance to disturbances inducing a change in expected difference between the signals.

15. The system of claim 13, wherein the shape of the target and the positional relationships of two or more coils relative to the joint's range of motion provide signals from their respective oscillator circuit(s) such that the signals change in approximately opposing manners with joint displacement; and
the computing unit(s) combines the signals by measuring a difference of the signals and using a result to:
determine the displacement of the joint, thereby providing resistance to disturbances inducing a similar change in the signals due to noise or nonidealities in the joint or drift in the oscillators due to temperature.

16. The system of claim 13, wherein the target shape and the positional relationship of two or more coils relative to the joint's range of motion provide continuous and periodic signals from their respective oscillator circuit(s) such that the signals, being continuous and periodic, no longer have a 1 to 1 mapping of signal to joint displacement and there is ambiguity within the period of such signals;
wherein the computing unit(s) combines the signals by:
using a relative positional relationships between the coils to determine a phase offset of the signals that respective oscillators produce; and
using the expected phase offset of the signals combined with the actual signal received to:
resolve the intra-period ambiguity of any one signal using the additional signal(s) to determine the displacement of the joint within a period.

17. The system of claim 16, wherein the computing unit(s) monitor the number of cycles of the periodic signal that have elapsed, counting whole or partial cycles of joint motion in one direction as positive, and whole or partial cycles of joint motion in the other direction as negative, such that the total displacement of the joint from a starting point can be determined; and
wherein the computing unit(s) receive an input to indicate the starting point as an absolute reference to displacement of the joint.

18. The system of claim 15, wherein the signals are combined to determine the displacement of the joint of the mechanism with the benefits of each such system in use.
